(19) United States
(12) Patent Application Publication
(10) Pub. No.: US 2025/0066695 A1
(43) Pub. Date: Feb. 27, 2025

(54) METHOD OF ENZYMATIC CLEANING TO REMOVE MICROORGANISMS OR BIOFILM FROM INDUSTRIAL EQUIPMENT

(71) Applicant: ECOLAB USA INC., St. Paul, MN (US)

(72) Inventors: Linna Wang, Shanghai (CN); Liang JI, Shanghai (CN); Jinsen Gao, Shanghai (CN)

(21) Appl. No.: 18/729,410

(22) PCT Filed: Jan. 20, 2022
(86) PCT No.: PCT/CN2022/072861

§ 371 Date: Jul. 16, 2024

(51) Int. Cl. C11D 3/386; C11D 3/48; C12Q 1/06; C12Q 1/22

(52) U.S. Cl. CPC C11D 3/38618; C11D 3/38627; C11D 3/48; C12Q 1/06; C12Q 1/22; C11D 2111/20

(57) ABSTRACT

The present disclosure provides methods for cleaning industrial equipment. In one example, a method for reducing microorganism from industrial equipment includes: (1) cleaning the equipment with one or more CIP compositions using a CIP process; (2) rinsing the equipment with water; (3) adding an enzyme composition into the CIP process; (4) analyzing a sample of the fluid within the CIP process before, after, or before and after the addition of the enzyme composition to detect microorganisms; and (5) modifying the CIP process in response to any detected microorganism.

### BACKGROUND

Clean-in-place (CIP) cleaning techniques are a specific cleaning regimen adapted for removing contaminants from the internal components of industrial equipment such as tanks, lines, pumps and other equipment used for processing typically liquid product streams such as beverages, milk, juices, corn stillage, ethanol, beer, wine, etc. These product streams leave soil deposits on the inside of the equipment that need to be removed. The soil deposits can include protein, fat, carbohydrate, and mineral deposits from the products themselves. These soils can provide an environment for microbial growth and those microorganisms can form an additional soil that needs to be removed, including vegetative bacteria, spores, and biofilms. CIP cleaning involves passing cleaning compositions or solutions through the system without dismantling any system components. The minimum CIP technique involves passing the cleaning solution through the equipment and then resuming normal processing.

Although conventional CIP techniques are effective to remove most soils or bulk particles, they are not always sufficient at removing all types of contaminants. In particular, microorganisms and biofilms derived therefrom can be difficult to remove completely from the equipment surface by the conventional CIP process. These unremoved contaminants can cause serious quality and safety issues. Because these residual microorganisms or biofilms are located on the interior surfaces of the equipment, a plant operator cannot visually assess or manually test the cleanliness of the equipment and to verify the microorganism or biofilm removal. Without a way to identify the presence of microorganisms within the CIP system, there is a risk that residual microorganisms and/or biofilms will remain on the interior of equipment surfaces and continue to grow. It is against this background that the present disclosure is made.

### SUMMARY

In one aspect, the present disclosure relates to a method for detecting or removing microorganisms and/or biofilm within industrial equipment, the method comprising: (1) cleaning the equipment with one or more CIP compositions using a CIP process; (2) rinsing the equipment with water; (3) adding an enzyme composition into the CIP process; and (4) adding to the CIP process a cleaning composition or an antimicrobial composition, or both. In some embodiments, the method further comprises: rinsing the equipment with water to remove the cleaning composition and/or the antimicrobial composition.

In certain embodiments, the antimicrobial composition comprises one or more antimicrobial agents selected from the group of peroxycarboxylic acids, phenolics, halogen compounds, quaternary ammonium compounds, metal derivatives, amines, alkanolamines, nitro derivatives, anilides, organosulfur compounds, sulfur-nitrogen compounds, ultraviolet light, electrolyzed water, and any combinations thereof.

In some embodiments, the enzyme composition comprises one or more enzymes selected from peroxidase, oxidoreductase, transferase, hydrolase, lyase, isomerase, ligase, protease, peptidase, lipase, esterase, amylase, polysaccharidase, carbohydrase, cellulase, hemicellulose, chitinase, glucanase, glycosidase, glucosidase, xylanase, mannanase, arabanase, DNase, RNase, phosphatase, phosphodiesterase, laccase, oxidoreductase, or combinations thereof.

In some embodiments, the enzyme composition further comprises one or more ingredients selected from enzyme stabilizer, salt, buffering agent, solvent, thickener, humectant, enzyme inhibitor, preservative, surfactant, dispersant, chelating agent, enzyme activity enhancer, or any combination thereof.

In some embodiments, the cleaning composition is an acid cleaning composition. In some embodiments, the cleaning composition includes an acid source selected from the group of mineral acids (e.g., phosphoric acid, nitric acid, sulfuric acid), organic acids (e.g., lactic acid, acetic acid, hydroxyacetic acid, citric acid, glutamic acid, glutaric acid, methane sulfonic acid, acid phosphonates, and gluconic acid), carboxylic acid, peroxyacids or peracids, oxygen-generating acids, or any combinations thereof. In some embodiments, the cleaning composition includes an acid source in an amount from about 0.01 wt % to about 99 wt %, or from about 0.1 wt % to about 50 wt %, or from about 1 wt % to about 25 wt %, by weight of the cleaning composition. In some embodiments, the cleaning composition has a pH from about 0 to about 6, or from about 1 to about 5, or from about 2 to about 4. In some embodiments, the cleaning compositions form a use solution when added to the equipment, and the use solution has a pH from about 1 to about 6, or from about 2 to about 5, or from about 3 to about 4.

In some embodiments, the cleaning composition is an alkaline cleaning composition. In some embodiments, the cleaning composition includes an alkalinity source in an amount from about 0.01% to about 99% by weight of the cleaning composition. In some embodiments, the fluid of the CIP process has a total amount of alkalinity source from about 5 ppm to about 25,000 ppm in the presence of the cleaning composition.

In some embodiments, the cleaning composition (acidic or alkaline) further comprises one or more antimicrobial agents. In some embodiments, the cleaning composition comprises one or more additives selected from oxidizing agent, chelating agent, sequestering agent, sanitizer or antimicrobial agent, dye, rheological modifier, gelling agent, thickener, pH modifiers, acid, base, preservative, processing aid, corrosion inhibitor, surfactant, dispersant, wetting agent, or any combinations thereof.

In some embodiments, the method comprises analyzing a sample of the fluid within the CIP process before, after, or before and after the addition of the enzyme composition to detect microorganisms within the fluid.

In some embodiments, the analyzing a sample of the enzyme solution comprises using an in-plate culture identification method. In some embodiments, the culture identification method comprises cultivating the sample on a cultural medium in a plate and performing a standard plate count for one or more microorganisms, wherein the microorganism is selected from the group of bacteria, yeast and mold, psychrophiles, psychrotrophs, mesopiles, thermophiles, aerobes, anaerobes, facultative anaerobes, bacteria spore former, spoilage microorganism, pathogens, specific biofilm indicator microorganism, or any combinations thereof.

In some embodiments, the analyzing a sample of the enzyme solution comprises using an ATP photometry method. In some embodiments, the ATP photometry method comprises measuring the ATP level of a sample using bioluminescence assay.

In some embodiments, the ATP photometry method comprises: extracting Adenosine monophosphate (AMP) and/or adenosine diphosphate (ADP) from the sample; converting the AMP and/or the ADP to ATP using one or more enzymes; and measuring the level of the converted ATP.

In some embodiments, the analyzing a sample of the enzyme solution comprises using one or more rapid microbial detection methods selected from the group of: a PCR-based rapid detection technique and/or a biosensor; an immunoassay; a bioburden test; a biofilm cell staining and subsequent optical quantification technique; a biofilm extracellular polymeric substances (EPS) staining and subsequent optical quantification technique, or any combinations thereof.

In some embodiments, the analyzing a sample of the enzyme solution comprises one or more steps selected from the group of measuring oxygen consumption, measuring CO₂ production, measuring production of metabolites, or any combinations thereof.

In some embodiments, the method further comprises a microorganism remediation step in response to any detected microorganism. In some embodiments, the microorganism remediation step is selected from the group of adjusting the concentration of the CIP composition, changing the chemistry of the CIP composition, adding additional CIP compositions, adding a cleaning composition to the CIP process, adding an antimicrobial composition to the CIP process, and combinations thereof.

In another aspect, the present disclosure relates to a method for verifying microorganism/biofilm elimination from industrial equipment, the method comprising: (1) cleaning the equipment with one or more CIP compositions using a CIP process; (2) rinsing the equipment with water; (3) adding an enzyme composition into the CIP process; and (4) analyzing a sample of fluid within the CIP process before, after, or before and after the addition of the enzyme composition to detect microorganisms within the CIP process.

In some embodiments, the method further includes deactivating enzyme(s) of the enzyme composition after analyzing the sample. In some embodiments, the method further includes rinsing the equipment with water to remove the enzyme composition.

In some embodiments, the method further includes adding a cleaning composition to the CIP process. In some embodiments, the method further includes adding an antimicrobial composition to the CIP process. In some embodiments, the method further comprises a microorganism remediation step in response to any detected microorganism.

In yet another aspect, the present disclosure relates to a method for reducing microorganisms or biofilm from industrial equipment, the method comprising: (1) cleaning the equipment with one or more CIP compositions using a CIP process; (2) rinsing the equipment with water; (3) adding an enzyme composition into the CIP process; (4) analyzing a sample of the fluid within the CIP process before, after, or before and after the addition of the enzyme composition to detect microorganisms; and (5) performing a remediation step in response to any detected microorganism.

In a further aspect, the present disclosure relates to a method for cleaning industrial equipment, the method comprising: (1) cleaning the equipment with one or more CIP compositions using a CIP process; (2) rinsing the equipment with water; (3) a first enzyme treatment step using a first enzyme composition to clean the equipment; (4) analyzing a sample of the fluid within the CIP process before, after, or before and after the enzyme treatment to detect microorganisms therein; (5) performing a first remediation step in response to any detected microorganism, and (6) rinsing the equipment with water to remove the composition(s) used in the remediation step.

In some embodiments, the method further includes: performing a second enzyme treatment using a second enzyme composition to clean the equipment. In some embodiments, the method further includes: analyzing a sample of the fluid within the CIP process after the second enzyme treatment; adding a cleaning composition or an antimicrobial composition, or both after the second enzyme treatment; or both. In some embodiments, the method further includes: performing a second remediation step in response to any detected microorganism after the second enzyme treatment, wherein the second remediation step includes one or more steps selected from the group of adding an additional step selected from the group of adjusting the concentration of the CIP composition, changing the chemistry of the CIP composition, adding additional CIP compositions, adding a cleaning composition to the CIP process, adding an antimicrobial composition to the CIP process, and combinations thereof. In some embodiments, the first enzyme composition and the second enzyme composition are the same, or different from each other.

In some embodiments, the methods according to the present disclosure result in at least 1 log reduction, or at least 2 log reduction, or at least 3 log reduction, or at least 5 log reduction of the microorganism population of the equipment compared to the microorganism population before the enzyme treatment step. In some embodiments, the methods according to the present disclosure result in at least 1 log reduction, or at least 2 log reduction, or at least 3 log reduction, or at least 5 log reduction of the microorganism population of the equipment compared to the microorganism population before the remediation step or before adding the cleaning composition or antimicrobial composition.

### BRIEF DESCRIPTION OF THE DRAWINGS

FIG. 1 is a schematic illustration of the general approaches adopted by the present disclosure.

FIG. 2 illustrates a block diagram of one example method 10 for cleaning industrial equipment, in accordance with various embodiments of the present disclosure.

FIG. 3 illustrates a block diagram of one example method 20 for verifying microorganism or biofilm removal, in accordance with various embodiments of the present disclosure.

FIG. 4 illustrates a block diagram of one example method 30 for cleaning industrial equipment, or verifying microorganism/biofilm removal, in accordance with various embodiments of the present disclosure.

FIG. 5 illustrates a block diagram of an example operation 500 of the method 30 shown in FIG. 4.

FIG. 6 illustrates a block diagram of an example operation 510 of the operation 500 shown in FIG. 5.

FIG. 7 illustrates a block diagram of another example method 40 for cleaning industrial equipment, in accordance with various embodiments of the present disclosure.

Although specific features of various embodiments may be shown in some drawings and not in others, this is for convenience only. Any feature of any drawing may be referenced and/or claimed in combination with any feature of any other drawing. Corresponding reference characters indicate corresponding parts throughout the drawings.

### DETAILED DESCRIPTION

The present disclosure provides compositions and methods generally related to cleaning industrial equipment using a CIP process. In some aspects, the present disclosure provides a method including one or more or all of the following steps: cleaning the equipment with one or more CIP compositions using a CIP process; rinsing the equipment with water; performing an enzyme treatment; adding an enzyme composition into the CIP process; analyzing a sample of fluid within the CIP process before, after, or before and after the addition of the enzyme composition to detect microorganisms within the CIP process; deactivating enzyme(s) of the enzyme composition; performing a remediation step; adding a cleaning composition to the CIP process; adding an antimicrobial composition to the CIP process; modifying the CIP composition in response to any detected microorganism; adjusting the concentration of the CIP composition, changing the chemistry of the CIP composition, adding additional CIP composition, adding additional cleaning composition to the CIP process, adding additional antimicrobial composition to the CIP process; rinsing the equipment to remove the enzyme(s) of the enzyme composition, or the cleaning composition/antimicrobial composition, or the compositions used in the remediation step; performing a second enzyme treatment after remediation step; analyzing a sample of the fluid within the CIP process before, after, or before and after the second enzyme treatment to detect microorganism or biofilm, performing a second remediation step after the second enzyme treatment; or repetition of any steps described herein.

The present methods provide a number of advantages. Frequently, industrial equipment to be cleaned by CIP has complex contaminants with layered configurations. For example, microorganisms and biofilm on the interior surface of the equipment may adhere directly to the surface, and one or more outer layers of mineral deposits or larger soil particles cover the microorganisms or biofilm layer and provide a protective barrier for the microorganisms or biofilm. The conventional CIP techniques or compositions usually can remove the exposed soil layer, bulk soils, or mineral deposits, but they may not reach the interior microorganisms or a biofilm layer, may fail to completely detach or remove the microorganisms or biofilm from the surface, or do not generate enough flow in certain areas to sufficiently disrupt the microorganisms or biofilm. Advantageously, the present methods include a separate enzyme treatment after the conventional CIP cleaning by adding an enzyme composition that can effectively reach the unremoved or residual or hidden microorganisms or biofilm remaining after the conventional CIP cleaning. The enzyme compositions of the present disclosure include one or more enzymes that can effectively penetrate the microorganisms or biofilm, weaken and soften the microorganisms or biofilm, break down the extracellular matrix or extracellular polymeric substances (EPS) derived from the biofilm, detach the microorganisms or biofilm from the surface, disperse or suspend the microorganisms or biofilm in the fluid within the CIP process.

It is noted that conventional cleaning solutions used in the CIP process are usually composed of harsh chemicals or used under extreme conditions (high temperature, extreme pH, etc.), which are typically not compatible with enzymes. For example, acid cleaner, alkaline cleaner, or bleach solution that are commonly used in conventional CIP process can seriously inhibit enzyme activity and undermine the effectiveness thereof. The separate enzyme treatment of the present methods allows the enzyme compositions to take effect under optimal conditions without the interference of harsh chemicals and therefore maximizes the performance of microorganisms or biofilm detachment and removal. Additionally, the multi-step process allows the bulk soils to be removed so that any microorganism or biofilm soils are exposed and more accessible for the enzymes to contact, breakdown, and remove.

Moreover, the present methods advantageously provide a surveillance step that allows for sampling and analyzing the fluid within the CIP process to detect or identify residual biofilm or microorganisms that are difficult to remove by conventional CIP cleaning. In particular, the enzyme treatment step allows for sampling so an operator can easily take a sample directly from the fluid before, during, or after the enzyme treatment. Analysis of the sample using rapid microorganism identification techniques provides qualitative or quantitative information about the biofilm type, microorganism species, metabolic state of the microorganism, and/or contamination level. Such information may provide valuable guidance or direction for subsequent cleaning, including changes to the normal CIP cleaning protocol or remediation steps.

Further, the present methods advantageously provide one or more remediation steps in response to the detected or identified microorganisms from the enzyme treatment step. The subsequent remediation step following the enzyme treatment step allows for eliminating microorganisms and/or biofilm by using a modified CIP composition or CIP condition, a cleaning composition, and/or an antimicrobial composition that targets and kills the specific microorganism detected and identified from the enzyme treatment step. The enzyme treatment, microorganism detection, and microorganism remediation could be repeated until the equipment is cleaned. Thus, the present methods provide users with a proof-of-clean of the equipment and validation of microorganism removal, which significantly reduces the risk of microorganism contamination and improves the overall efficiency of the CIP process.

The embodiments of this disclosure are not limited to particular methods for microorganism or biofilm detection and cleaning, which can vary and are understood by skilled artisans. It is further to be understood that all terminology used herein is for the purpose of describing particular embodiments only and is not intended to be limiting in any manner or scope. For example, all units, prefixes, and symbols may be denoted in its SI accepted form. Numeric ranges recited within the disclosure are inclusive of the numbers defining the range and include each integer within the defined range.

### Definitions and Interpretations of Selected Terms

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

As used herein, “weight percent,” “wt %, “percent by weight,” “% by weight,” and variations thereof refer to the concentration of a substance as the weight of that substance divided by the total weight of the composition and multiplied by 100. It is understood that, as used here, “percent,” “%,” and the like are intended to be synonymous with “weight percent,” “wt %,” etc.

It should be noted that, as used in this disclosure and the appended claims, the singular forms “a,” “an,” and “the” include plural referents unless the content clearly dictates otherwise. Thus, for example, reference to a composition containing “a compound” includes a composition having two or more compounds. It should also be noted that the term “or” is generally employed in its sense including “and/or” unless the content clearly dictates otherwise.

As used herein, the term “about” is used in conjunction with numeric values to include normal variations in measurements as expected by persons skilled in the art, and is understood to have the same meaning as “approximately” and to cover a typical margin of error, such as +15%, +10%, +5%, +1%, ±0.5%, or even +0.1% of the stated value. The term “about” also encompasses amounts that differ due to different equilibrium conditions for a composition resulting from a particular initial composition. Whether or not modified by the term “about,” the claims include equivalents to the quantities.

The term “cleaning,” as used herein, means to perform or aid in soil removal, bleaching, microbial population reduction, or combination thereof. For the purpose of this patent application, successful microbial reduction is achieved when the microbial populations are reduced by at least about 50%, or by significantly more than is achieved by a wash with water. Larger reductions in microbial population provide greater levels of protection.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

As used herein, the term “biofilm” means an extracellular matrix in which a population of microorganisms are dispersed and/or form colonies. Biofilms are understood to be typically made of extracellular polysaccharides, proteins, lipids, and DNA, often referred to as extracellular polymeric substances (EPS), that are concentrated at an interface (usually solid/liquid) and act as a binding agent that surrounds such populations of microorganisms. Biofilms are further understood to include complex associations of cells, extracellular products and detritus (or non-living particulate organic material) that are trapped within the biofilm or released from cells within the biofilm. The term biofilm, as used herein, further refers to the ASTM definition of biofilm as an accumulation of bacterial cells immobilized on a substratum and embedded in an organic polymer matrix of microbial origin. Biofilms are understood to be a dynamic, self-organized accumulation of microorganisms and microbial and environmental by-products that is determined by the environment in which it lives.

According to the disclosure, the phrases “microorganism and/or biofilm remediation,” “removing microorganism and/or biofilm,” “reducing microorganism and/or biofilm” and like phrases, shall mean a reduction in the rate or extent of microorganism and/or biofilm growth, removal of existing microorganism and/or biofilm or portions of microorganism and/or biofilm on surfaces and/or eradication of existing microorganism and/or biofilm on a treated surface. According to the present disclosure, the compositions and methods disclosed herein may physically remove and kill microorganism and/or biofilm.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allowing a composition to be substantially free of that expressly stated ingredient.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

As used herein, the terms “increase,” “increasing,” “increased,” “improved,”, “improving,” “improvement”,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

### CIP Process and Industrial Equipment

In some aspects, the methods of the present disclosure apply to industrial equipment generally cleaned using clean-in-place (i.e., CIP) cleaning procedures. Non-limiting examples of such industrial equipment include evaporators, heat exchangers (including tube-in-tube exchangers, direct steam injection, and plate-in-frame exchangers), heating coils (including steam, flame or heat transfer fluid heated) re-crystallizers, pan crystallizers, spray dryers, drum dryers, tanks, filtration media, separation media, membranes, reverse osmosis membranes, nanofiltration membranes, ultrafiltration membranes, microfiltration membranes, seals, gaskets, and the pipes and pumps connecting the equipment together.

The methods of the present disclosure can be used generally to clean equipment that has been contaminated with any type of soils. In some embodiments, the method can be used to clean equipment that has been contaminated with thermally degraded soils, i.e., caked on soils or burned on soils, such as proteins or carbohydrates. Exemplary thermally degraded soils include food soils that have been heated during processing, e.g., dairy products heated on pasteurizers. In some embodiments, the compositions and methods of the present disclosure can also be used to clean equipment that has been contaminated with non-thermally degraded soils that are not easily removed completely using conventional cleaning techniques. The methods of the present disclosure provide enhanced cleaning of these hard to remove soil types and a proof-of-clean for the cleaning process.

In particular, the methods of the present disclosure can be used to detect and remove microorganism, biofilm, or fouled biomaterial from a surface of industrial equipment. For example, separation media such as membranes are typical industrial equipment in various industries. During operation, the membranes gradually become fouled. In particular, microorganism/biofilm growth and mineral deposits can accumulate on membranes. The biofilm may contain particulates that are substantially smaller in size comparing with bulk soils. As discussed above, a typical contaminant on the surface of an industrial equipment may have multiple layers. The biofilm layer usually adheres directly to the surface, and one or more outer layers of mineral deposits or larger soil particles cover the biofilm layer, or entrapped within biofilm layer. The present methods are particular useful to detect and remove microorganisms or a biofilm layer that is hidden behind the soil layer or invisible in visual inspection.

Exemplary industries in which the methods of the present disclosure can be used include, but are not limited to: the food and beverage industry, e.g., the dairy, cheese, sugar, and brewery industries; oil processing industry; industrial agriculture and ethanol processing; and the pharmaceutical manufacturing industry. Non-limiting examples of industrial plants where the present methods are applicable include dairy plants, biofuel plants, brewery plants, food-processing plants, farming plants, paper-making plants, gas and oil plants, and coolant plants.

### Compositions and Ingredients

The present disclosure is related to compositions and methods of using the compositions for cleaning industrial equipment using a CIP process. The methods are used for one or more of the following purposes: detecting or removing microorganism and/or biofilm within the industrial equipment; verifying microorganism and/or biofilm elimination from industrial equipment; reducing microorganism and/or biofilm from industrial equipment; improving efficiency of the CIP process; and providing a proof-of-clean of the CIP process.

In general, the present methods include one or more operations or steps, and various compositions or formulations are used in the present methods or a step or operation thereof. In one particular embodiment, the present method includes one or more of the following steps: cleaning the equipment with one or more CIP compositions using a CIP process; rinsing the equipment with water; adding an enzyme composition into the CIP process; analyzing a sample of fluid within the CIP process before, after, or before and after the addition of the enzyme composition to detect microorganisms within the CIP process; adding a cleaning composition to the CIP process; adding an antimicrobial composition to the CIP process; rinsing the equipment with water to remove the cleaning composition and/or the antimicrobial composition; modifying the CIP composition in response to any detected microorganism; adjusting the concentration of the CIP composition, changing the chemistry of the CIP composition, adding additional CIP composition, adding additional cleaning composition to the CIP process, and adding additional antimicrobial composition to the CIP process. In some embodiments, the present method includes repeating one or more of the steps described herein.

In some embodiments, the present methods and a step or operation thereof involve using one or more CIP compositions, one or more enzyme compositions, one or more cleaning compositions, and one or more antimicrobial compositions, one or more deactivating enzyme(s) of the enzyme composition. Each of the compositions used in the present methods may contain one or more active ingredients. In some embodiments, the compositions used in the present methods contain a carrier. The compositions of the present methods when added to the CIP process may form a use solution within the industrial equipment. In some embodiments, the compositions of the present methods are a concentrate. Non-limiting embodiments and examples of the compositions, ingredients, and carrier used in the present disclosure are described in the following passages.

### CIP Compositions

In some embodiments, the present method includes cleaning the equipment with one or more CIP compositions using a CIP process. The CIP process used in the present method includes conventional CIP techniques. The CIP process includes applying one or more CIP compositions (typically derived from a concentrate having about 0.1-10 wt % of the total functional components) onto the surface of the industrial equipment to be cleaned. The CIP composition flows across the surface (1 to 10 feet/second), slowly removing the soil. Either a new or modified CIP composition is re-applied to the surface, or the same CIP composition is recirculated and re-applied to the surface.

Examples of the CIP composition include an acid solution, an alkaline solution, and a neutral solution. In some embodiments, CIP cleaning is performed by adding an alkaline solution (e.g., with sodium hydroxide) as the CIP composition to the equipment followed by rinsing with water. In other applications, multiple CIP compositions may be used in the CIP process simultaneously, separately, or in a coordinated fashion. For example, a CIP process to remove a soil (including organic, inorganic, or a mixture of the two components) includes at least three treatments: an alkaline treatment using a first CIP composition comprising an alkaline solution, an acid treatment using a second CIP composition comprising an acid solution, and a rinsing with a fresh water. The alkaline solution softens the soils and removes the organic, alkaline-soluble soils. The subsequent acid solution removes mineral soils left behind by the alkaline cleaning step. The strength of the alkaline and acid solutions and the duration of the cleaning steps are typically dependent on the durability of the soil and the nature of the materials being cleaned. The water rinse removes any residual solution and soils and cleans the surface prior to the equipment being returned on-line. In some embodiments, the acid or alkaline treatments and the rinsing step may be repeated, and the CIP compositions used in the repeating steps may be modified and differentiated from the originally used CIP compositions in concentration, ingredients, amount, and treatment time.

In some embodiments, the CIP process of the present method may include two steps that work with each other to generate a chemical reaction within the equipment and within the soil. For example, U.S. Patent Publication No. 2019/0039102 describes a gas-generating process using a pretreatment solution and an override solution. The pretreatment solution penetrates the soil and the override solution is applied after the pretreatment solution to activate the pretreatment solution. In some embodiments, the combination of pretreatment and override solutions generates gas on and in the soil, providing a soil disruption effect. This soil disruption effect has been found to facilitate and enhance the cleaning of these types of soils compared with conventional cleaning techniques. U.S. Patent Publication No. 2019/0039102 is incorporated by reference herein in its entirety.

In some embodiments, the CIP composition used in the present methods is an acid cleaner, acid solution, or an acid cleaning composition. The acid cleaner generally includes one or more inorganic and organic acids. Exemplary acid sources suitable for use with the methods of the present disclosure include, but are not limited to, mineral acids (e.g., phosphoric acid, nitric acid, sulfuric acid) and organic acids (e.g., lactic acid, acetic acid, hydroxyacetic acid, citric acid, glutamic acid, glutaric acid, methane sulfonic acid, acid phosphonates (e.g., HEDP), and gluconic acid).

The alkaline cleaner according to the present disclosure includes at least one alkaline source. Exemplary alkaline sources suitable for use with the methods of the present disclosure include, but are not limited to, basic salts, amines, alkanolamines, alkali metal hydroxides, carbonates, bicarbonates, and silicates. More specific alkaline sources include NaOH (sodium hydroxide), KOH (potassium hydroxide), TEA (triethanolamine), DEA (diethanolamine), MEA (monoethanolamine), sodium carbonate, and morpholine, sodium metasilicate and potassium silicate. In some embodiments, the alkaline source selected is compatible with the surface to be cleaned.

In some embodiments, CIP cleaning step of the present disclosure involves using a neutral CIP composition. The neutral CIP composition or a use solution thereof may have a neutralized medium with a pH in a range from about 4-10. The neutral CIP compositions may include one or more of the following ingredients: a buffer, a weak acid, a weak base, a surfactant. One example of the neutral CIP composition is a neutral peroxycarboxylic acid comprising a peroxycarboxylic acid. Peroxycarboxylic (or percarboxylic) acids generally have the formula R(CO₃H)ₙ, where, for example, R is an alkyl, arylalkyl, cycloalkyl, aromatic, or heterocyclic group, and n is one, two, or three, and named by prefixing the parent acid with peroxy. The R group can be saturated or unsaturated as well as substituted or unsubstituted. The composition and methods of the disclosure can employ peroxycarboxylic acids containing, for example, 6 to 12 carbon atoms. For example, peroxycarboxylic (or percarboxylic) acids can have the formula R(CO₃H)ₙ, where R is a C₁-C₂₄ alkyl group, a C₅-C₂₄ cycloalkyl, a C₅-C₂₄ arylalkyl group, C₁-C₂₄ aryl group, or a C₅-C₂₄ heterocyclic group; and n is one, two, or three. Examples of neutral peroxycarboxylic acid compositions for CIP process can be found in U.S. Patent Publication No. 20090200234, which is incorporated herein by reference in its entirety.

The CIP composition may optionally include a bleaching agent. Bleaching agents include bleaching compounds capable of liberating an active halogen species, such as Cl₂, Br₂, —OCl— and/or —OBr, under conditions typically encountered during the cleansing process. Suitable bleaching agents include, for example, chlorine-containing compounds such as a chlorine, a hypochlorite, chloramine. Preferred halogen-releasing compounds include the alkali metal dichloroisocyanurates, chlorinated trisodium phosphate, the alkali metal hypochlorites, monochloramine and dichloramine, and the like. A bleaching agent may also be a peroxygen or active oxygen source such as hydrogen peroxide, perborates, sodium carbonate peroxyhydrate, phosphate peroxyhydrates, potassium permonosulfate, and sodium perborate mono and tetrahydrate, with and without activators such as tetraacetylethylene diamine, and the like.

The CIP composition can also include additional and optional ingredients, such as acidulant, surfactant, solvent, sequestrant, or mixtures thereof.

In some embodiments, the CIP cleaning treatment and/or the CIP composition described herein is free or substantially free from an enzyme.

### Enzyme Compositions

In some embodiments, the present method includes an enzyme treatment step by adding an enzyme composition into the CIP process after the conventional CIP steps. Before the enzyme composition is added, the CIP composition may be cleaned off by rinsing the industrial equipment with water. Most soils, soil particles, mineral deposits, and bulk contaminants are removed by the conventional CIP steps. Any microorganism contamination, including hidden biofilm or residual biofilm, that is not removed by the conventional CIP steps, remain on or attached to the surface of the industrial equipment. The enzyme composition added to the CIP process may form a use solution that can penetrate the microorganism contamination (including any biofilm) and initiate enzymatic reaction or interaction. The microorganism treated by the enzymatic composition can become fragile, weakened, degraded, softened, and detached from the surface of the industrial equipment. The microorganism may comprise a variety of microorganisms or pathogens, such as bacteria or fungi, both gram positive and negative bacteria, including for example Pseudomonas aeruginosa, Escherichia coli, Staphylococcus epidermidis, Staphylococcus aureus and Listeria monocytogenes. The microorganism may be in a vegetative or spore state. The microorganism may also be part of a biofilm.

The present enzyme composition may be made from a commercial enzyme product such as enzyme products sold by the company Novozyme®. The commercial enzyme product may be in a form of concentrate, solid, or powder. The commercial enzyme product provides a source of active enzymes but may also include other additives to maintain the active enzymes during manufacturing, shipping, and storage of the commercial enzyme product.

In some embodiments, the enzyme composition has a total enzyme amount from about 0.001 wt % to about 10 wt %, or from about 0.01 wt % to about 5 wt %, or from about 0.1 to about 2 wt %, based on the weight of the enzyme composition.

In some embodiments, the enzyme composition added to the CIP process forms a use solution that has a total enzyme amount in a range from about 10 ppm to about 10,000 ppm, or from about 50 ppm to about 1000 ppm, or from about 100 ppm to about 500 ppm.

In some embodiments, the use solution of the added enzyme composition has a pH value from about 2 to about 12, or from about 4 to about 10, or from about 7 to about 10.

In some embodiments, the enzyme treatment using the added enzyme composition or the use solution thereof is performed at a temperature of about 30° C. to about 85° C., or from about 40° C. to about 70° C., or from about 50° C. to about 60° C. within the CIP process.

In some embodiment, the enzyme treatment using the enzyme composition in the present disclosure last for at least about 5 minutes, at least about 10 minutes, at least 30 minutes, at least 90 minutes, at least 120 minutes, or at least 150 minutes.

In some embodiments, the enzyme composition provides at least 50% reduction, or at least 90% reduction, or at least 99% reduction of the microorganism population of the equipment compared to the microorganism population before the enzyme treatment.

### Enzymes

Non-limiting examples of suitable enzymes used in the enzyme composition include peroxidase, oxidoreductase, transferase, hydrolase, lyase, isomerase, ligase, protease, peptidase, lipase, esterase, amylase, polysaccharidase, carbohydrase, cellulase, hemicellulose, chitinase, glucanase, glycosidase, glucosidase, xylanase, mannanase, arabanase, DNase, RNase, phosphatase, phosphodiesterase, laccase, oxidoreductase, or combinations thereof. The choice of enzyme(s) takes into account factors such as type of soil, type of microorganism, type of biofilm, nature of the CIP application, size and function of the equipment, the equipment material, pH-activity, stability optima, thermostability, stability, chelants, builders, etc.

A valuable reference on enzymes is “Industrial Enzymes,” Scott, D., in Kirk-Othmer Encyclopedia of Chemical Technology, 3rd Edition, (editors Grayson, M. and EcKroth, D.) Vol. 9, pp. 173-224, John Wiley & Sons, New York, 1980, which is incorporated herein by reference in its entirety.

In some embodiments, the enzyme composition of the present disclosure includes two or more enzymes.

### Protease

A protease can be derived from a plant, an animal, or a microorganism. Preferably the protease is derived from a microorganism, such as a yeast, a mold, or a bacterium. Preferred proteases include serine proteases active at alkaline pH, preferably derived from a strain of Bacillus such as Bacillus subtilis or Bacillus licheniformis; these preferred proteases include native and recombinant subtilisins. The protease can be purified or a component of a microbial extract, and either wild type or variant (either chemical or recombinant). Examples of proteolytic enzymes include (with trade names) Savinase®; a protease derived from Bacillus lentus type, such as Maxacal®, Opticlean®, Durazym®, and Properase®; a protease derived from Bacillus licheniformis, such as Alcalase® and Maxatase®; and a protease derived from Bacillus amyloliquefaciens, such as Primase®. Preferred commercially available protease enzymes include those sold under the trade names Alcalase®, Savinase®, Primase®, Durazym®, Esperase®, Maxatase®, Maxacal®, Maxapem®, Purafect®, Purafect OX, Properase, Opticlean® or Optimase®, Progress Uno™, Progress Excel™, Liquanase™, Blaze Evity™, Everlase™, Polarzyme™, Relase™, Coronase™, and the like. A mixture of such proteases can also be used.

### Amylase

An amylase can be derived from a plant, an animal, or a microorganism. Preferably the amylase is derived from a microorganism, such as a yeast, a mold, or a bacterium. Preferred amylases include those derived from a Bacillus, such as B. licheniformis, B. amyloliquefaciens, B. subtilis, or B. stearothermophilus. The amylase can be purified or a component of a microbial extract, and either wild type or variant (either chemical or recombinant), preferably a variant that is more stable under washing or presoak conditions than a wild type amylase.

Examples of amylase enzymes that can be employed include those sold under the trade name Stainzyme®, Rapidase, Termamyl®, Fungamyl® Duramyl®, Purastar STL, Purastar OXAM, and the like. Preferred commercially available amylase enzymes include the stability enhanced variant amylase sold under the trade name Duramyl®. A mixture of amylases can also be used.

### Cellulases

A suitable cellulase can be derived from a plant, an animal, or a microorganism. Preferably the cellulase is derived from a microorganism, such as a fungus or a bacterium. Preferred cellulases include those derived from a fungus, such as Humicola insolens, Humicola strain DSM1800, or a cellulase 212-producing fungus belonging to the genus Aeromonas and those extracted from the hepatopancreas of a marine mollusk, Dolabella Auricula Solander. The cellulase can be purified or a component of an extract, and either wild type or variant (either chemical or recombinant).

### Lipases

A suitable lipase can be derived from a plant, an animal, or a microorganism. Preferably the lipase is derived from a microorganism, such as a fungus or a bacterium. Preferred lipases include those derived from a Pseudomonas, such as Pseudomonas stutzeri ATCC 19.154, or from a Humicola, such as Humicola lanuginosa (typically produced recombinantly in Aspergillus oryzae). The lipase can be purified or a component of an extract, and either wild type or variant (either chemical or recombinant). Examples of lipase enzymes include those sold under the trade names Lipase P “Amano” or “Amano-P”, Lipolase®, Amano-CES, lipases derived from Chromobacter viscosum, e.g. Chromobacter viscosum var. lipolyticum NRRLB 3673, lipases derived from Chromobacter viscosum lipases, and lipases derived from Pseudomonas gladioli or from Humicola lanuginosa A mixture of lipases can also be used.

Other enzymes that may be used in the present enzyme composition include but are not limited to cutinase enzymes, peroxidases such as horseradish peroxidase, ligninase, and haloperoxidases such as chloro- or bromo-peroxidase, carbohydrases such as mannanase, pectate lyase, cyclomaltodextrin, glucanotransferase, xyloglucanase, bleaching enzymes such as peroxidases, laccases, oxygenases, lipoxygenase, (non-heme) haloperoxidases, endoglucanases, aminopeptidase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidoreductases, pectinolytic enzyme, peptidoglutaminase, peroxidase, phytase, polyphenoloxidase, proteolytic enzyme, ribonuclease, transglutaminase, or xylanase.

Commercially available enzyme compositions used in the present disclosure include Soluscope® products from Ecolab.

### Enzyme Stabilizer

The enzyme composition can optionally include one or more enzyme stabilizers. Exemplary enzyme stabilizers include calcium compounds, magnesium compounds, boron compounds and substituted boric acids, aromatic borate esters, peptides and peptide derivatives, polyols, low molecular weight carboxylates, relatively hydrophobic organic compounds (e.g. certain esters, diakyl glycol ethers, alcohols or alcohol alkoxylates), alkyl ether carboxylate in addition to a calcium ion source, benzamidine hypochlorite, lower aliphatic alcohols and carboxylic acids, N,N-bis(carboxymethyl) serine salts; (meth)acrylic acid-(meth)acrylic acid ester copolymer and PEG; lignin compound, polyamide oligomer, glycolic acid or its salts; poly-hexa-methylene-bi-guanide or N,N-bis-3-amino-propyl-dodecyl amine or salt; and mixtures thereof. Enzyme stabilizers are present from about 1 to about 30, or from about 2 to about 20, or from about 5 to about 15, or from about 8 to about 12, millimoles of stabilizer ions per liter of the enzyme composition as a concentrate. In some embodiments, enzyme stabilizers are present in a use solution of the enzyme composition with a concentration from about 0.01 to about 20, or from about 0.1 to about 10, or from about 0.1 to about 5, millimoles of stabilizer ions per liter of the enzyme composition as a use solution.

### Solvent

The enzyme composition can optionally include one or more solvents. Suitable solvents include organic and aqueous solvents. For example, suitable organic solvents include isopropanol, other lower alcohols, glycol ethers, mixtures thereof, or the like. For example, suitable aqueous solvents include water, mixtures of water with the organic solvent, mixtures thereof, or the like. In an embodiment, the solvent includes isopropanol, water, or a mixture thereof. The solvent can be present in the enzyme composition at about 0.01 to about 20 wt %, about 0.1 to about 10 wt %, about 0.5 to about 5 wt %, about 0.01 to about 1.0 wt %. The solvent (particularly a solvent like water, which can be employed as a diluent) can be present in the composition at about 0.01 to about 99 wt %, about 0.1 to about 99 wt %, about 1 to about 80 wt %, or about 10 to about 70 wt %.

### Surfactants

A surfactant or mixture of surfactants can be present in the composition or use solution of the present disclosure, including the CIP composition, the enzyme composition, the cleaning composition, and the antimicrobial composition. Examples of suitable surfactants include nonionic, cationic, and anionic surfactants. Examples of surfactants can be found in U.S. Pat. No. 10,433,547, the relevant part of which is incorporated herein by reference.

The enzyme composition according to the present disclosure may optionally include one or more additive or functional ingredients including but not limited to: buffering agent, pH-modifier, viscosity-modifier, thickener or gelling agent, humectants, antiredeposition agent, dispersant, chelating agent, preservative, or any combinations thereof. The enzyme composition of the present disclosure may further include one or more ingredients described elsewhere in the present disclosure.

### Cleaning Compositions

In some embodiments, the present method includes a microorganism or biofilm remediation step comprising adding a cleaning composition to the CIP process after the enzyme treatment. In the present methods, the bulk soils are removed by the CIP compositions leaving any remaining microorganisms or biofilm exposed and vulnerable to the enzyme composition. The addition of the enzyme composition breaks down any microorganism or biofilm soil, helps loosen it from the equipment surface, and disrupts the biofilm matrix. The addition of the cleaning composition after the enzyme composition helps to further remove any microorganism or biofilm soil and flush it from the system. The cleaning composition may be one of the CIP compositions that has been used in the CIP steps prior to the enzyme treatment. Alternatively, the cleaning composition may be a modification of the CIP composition used prior to the enzyme treatment with adjusted or optimized conditions such as concentration, ingredient, pH, ion strength, viscosity, amount, operating temperature, time of treatment, etc.

In some embodiments, the cleaning composition is different from the CIP composition. For example, the cleaning composition may contain a new ingredient that has not been used in the CIP composition prior to enzyme treatment. The new ingredient may be used to specifically target the type of microorganism if the microorganism was identified from the enzyme treatment. For example, the cleaning composition includes an antimicrobial agent that specifically kills the detected microorganism within the CIP process.

In some embodiments, the cleaning composition is an acid cleaning composition comprising an acid cleaner described supra. In some embodiments, the cleaning composition includes an acid source in an amount from about 0.01 wt % to about 99 wt %, or from about 0.1 wt % to about 50 wt %, or from about 1 wt % to about 25 wt %, by weight of the cleaning composition. In some embodiments, the cleaning composition has a pH from about 0 to about 6, or from about 1 to about 5, or from about 2 to about 4. In some embodiments, the cleaning composition forms a use solution when added to the equipment, and the use solution has a pH from about 1 to about 6, or from about 2 to about 5, or from about 3 to about 4.

In some embodiments, the cleaning composition is an alkaline cleaning composition comprising an alkaline source described supra. In some embodiments, the cleaning composition includes an alkalinity source in an amount from about 0.01 wt % to about 99 wt %, or from about 0.1 wt % to about 50 wt %, or from about 1 wt % to about 25 wt %, by weight of the cleaning composition. In some embodiments, the cleaning composition when added to the CIP process forms a use solution, and the use solution has a total amount of alkalinity source from about 5 ppm to about 25,000 ppm, or from about 100 ppm to about 10,000 ppm, or from about 500 μm to about 5,000 ppm.

### Antimicrobial Agents

The cleaning compositions of the present disclosure may optionally include an antimicrobial agent. Antimicrobial agents are chemical compositions that can be used in the composition to reduce microbial contamination. Generally, these materials fall in specific classes including peroxycarboxylic acids, phenolics, halogen compounds, quaternary ammonium compounds, metal derivatives, amines, alkanol amines, nitro derivatives, anilides, organosulfur and sulfur-nitrogen compounds, oxidizing antimicrobials, non-oxidizing antimicrobials, and miscellaneous compounds.

Common antimicrobial agents that may be used include phenolic antimicrobials such as pentachlorophenol, orthophenylphenol; halogen containing antibacterial agents that may be used include chlorine, chlorine dioxide, sodium hypochlorite, acidified sodium chlorite, sodium trichloroisocyanurate, sodium dichloroisocyanurate (anhydrous or dihydrate), iodine-poly(vinylpyrolidin-onen) complexes, bromine compounds such as 2-bromo-2-nitropropane-1,3-diol; quaternary antimicrobial agents such as benzalconium chloride, cetylpyridiniumchloride; amines and nitro containing antimicrobial compositions such as hexahydro-1,3,5-tris(2-hydroxyethyl)-s-triazine, dithiocarbamates such as sodium dimethyldithiocarbamate, peroxygen compounds such as peroxyacetic acid, peroxyformic acid, peroxyoctanoic acid, and a variety of other materials known in the art for their microbial properties. In a preferred embodiments, the antimicrobial agent includes a peroxygen compound such as peroxyacetic acid or peroxyoctanoic acid. Antimicrobial agents may be encapsulated to improve stability and/or to reduce reactivity with other materials.

When an antimicrobial agent is incorporated into the cleaning composition, it is preferably included in an amount of from about 0.01 wt % to about 5 wt %, from about 0.01 wt % to about 2 wt %, or from about 0.1 wt % to about 1.0 wt %.

In an embodiment, the antimicrobial agent described herein will provide at least a 1 log reduction, or at least a 2 log reduction, or 3 log reduction, or at least a 5 log reduction of a microorganism population or biofilm according to the present disclosure.

### Carrier

In some embodiments, the cleaning compositions include a carrier. A carrier in the disclosed compositions can be water, an organic solvent, or a combination of water and an organic solvent. The organic solvent can be an alcohol, a hydrocarbon, a ketone, an ether, an alkylene glycol, a glycol ether, an amide, a nitrile, a sulfoxide, an ester, or a combination thereof. Examples of suitable organic solvents include, but are not limited to, methanol, ethanol, propanol, isopropanol, butanol, 2-ethylhexanol, hexanol, octanol, decanol, 2-butoxyethanol, methylene glycol, ethylene glycol, 1,2-propylene glycol, 1,3-propylene glycol, diethyleneglycol monomethyl ether, diethylene glycol monoethyl ether, ethylene glycol monobutyl ether, ethylene glycol dibutyl ether, pentane, hexane, cyclohexane, methylcyclohexane, heptane, decane, dodecane, diesel, toluene, xylene, heavy aromatic naphtha, cyclohexanone, diisobutylketone, diethyl ether, propylene carbonate, N-methylpyrrolidinone, N,N-dimethylformamide, or a combination thereof.

The cleaning compositions of the present disclosure can comprise from about 1 wt % to about 80 wt %, from about 1 wt % to about 70 wt %, from about 1 wt % to about 60 wt %, from about 1 wt % to about 50 wt %, from about 1 wt % to about 40 wt %, from about 1 wt % to about 30 wt %, from about 1 wt % to about 20 wt %, from about 1 wt % to about 10 wt %, or any value there between of the one or more carrier, based on total weight of the cleaning composition.

### Builders

The cleaning compositions can optionally include a builder or mixture of builders. Builders include chelating agents (chelators), sequestering agents (sequestrants), and the like. The builder often stabilizes the composition or solution.

Builders and builder salts can be inorganic or organic. Examples of builders suitable for use with the methods of the present disclosure include, but are not limited to, phosphonic acids and phosphonates, phosphates, aminocarboxylates and their derivatives, pyrophosphates, polyphosphates, ethylenediamene and ethylenetriamene derivatives, hydroxyacids, and mono-, di-, and tri-carboxylates and their corresponding acids. Other builders include aluminosilicates, nitroloacetates and their derivatives, and mixtures thereof. Still other builders include aminocarboxylates, including salts of hydroxyethylenediaminetetraacetic acid (HEDTA), and diethylenetriaminepentaacetic acid. In some embodiments, a biodegradable aminocarboxylate or derivative thereof is present as a builder in the methods of the present disclosure. Non-limiting examples of biodegradable aminocarboxylate include methylglycinediacetic acid (MDGA) or a salt thereof, glutamic acid N,N-diacetic acid (GLDA) or a salt thereof.

In some embodiments, an organic chelating agent is used. Organic chelating agents include both polymeric and small molecule chelating agents. Organic small molecule chelating agents are typically organocarboxylate compounds or organophosphate chelating agents. Polymeric chelating agents commonly include polyanionic compositions such as polyacrylic acid compounds. Small molecule organic chelating agents include N-hydroxyethylenediaminetriacetic acid (HEDTA), ethylenediaminetetraacetic acid (EDTA), nitrilotriaacetic acid (NTA), diethylenetriaminepentaacetic acid (DTPA), ethylenediaminetetraproprionic acid triethylenetetraaminehexaacetic acid (TTHA), and the respective alkali metal, ammonium and substituted ammonium salts thereof. Aminophosphonates are also suitable for use as chelating agents with the methods of the disclosure and include ethylenediaminetetramethylene phosphonates, nitrilotrismethylene phosphonates, and diethylenetriamine-(pentamethylene phosphonate) for example. These aminophosphonates commonly contain alkyl or alkenyl groups with less than 8 carbon atoms.

Other suitable sequestrants include water soluble polycarboxylate polymers. Such homopolymeric and copolymeric chelating agents include polymeric compositions with pendant (—CO₂H) carboxylic acid groups and include polyacrylic acid, polymethacrylic acid, polymaleic acid, acrylic acid-methacrylic acid copolymers, acrylic-maleic copolymers, hydrolyzed polyacrylamide, hydrolyzed methacrylamide, hydrolyzed acrylamide-methacrylamide copolymers, hydrolyzed polyacrylonitrile, hydrolyzed polymethacrylonitrile, hydrolyzed acrylonitrile methacrylonitrile copolymers, or mixtures thereof. Water soluble salts or partial salts of these polymers or copolymers such as their respective alkali metal (for example, sodium or potassium) or ammonium salts can also be used. Preferred polymers include polyacrylic acid, the partial sodium salts of polyacrylic acid or sodium polyacrylate.

In some embodiments, the cleaning composition is an alkaline cleaning composition having a total amount of builder in a range from about 0.001 wt % to about 5 wt %, or from about 0.005 wt % to about 0.1 wt %, or from about 0.05 wt % to about 2.5 wt %, based on the total weight of the cleaning composition.

In some embodiments, the cleaning composition of the present disclosure may further include one or more additives including but not limited to a dye or odorant, a defoaming agent oxidizing agent, a preservative, a processing aid, a corrosion inhibitor, a dispersant, or any combinations thereof.

The cleaning composition may further comprise an ingredient described elsewhere in the present disclosure.

In some embodiments, the cleaning composition is an antimicrobial composition described herein. In some embodiments, the cleaning composition is an enzyme composition described herein. In some embodiments, the cleaning composition used herein provides at least 1 log reduction, or at least 2 log reduction, or at least 3 log reduction, or at least 5 log reduction of the microorganism population of the equipment before adding cleaning composition.

### Antimicrobial Compositions

In some embodiments, the method of the present disclosure includes an antimicrobial treatment using an antimicrobial composition. The antimicrobial treatment may be included in the microorganism/biofilm remediation step. For example, if certain type of biofilm or class of microorganism has been determined or identified during the enzyme treatment step, an antimicrobial composition may be added to the CIP process after the enzyme treatment step to specifically target and kill the identified microorganism of the biofilm to be removed. The antimicrobial composition may be used in combination with the cleaning composition, or in a separate step before or after treatment with the cleaning composition, or in a coordinated fashion (e.g., in an alternating manner) with the cleaning composition.

The antimicrobial composition of the present disclosure comprises one or more antimicrobial agents, and optionally an antimicrobial solvent, an additional antimicrobial agent, a cleaning composition, each of which is described supra. In some embodiments, the antimicrobial composition further comprises an additive or additional functional ingredient described elsewhere according to the present disclosure. In some embodiments, the antimicrobial composition used herein provides at least 1 log reduction, or at least 2 log reduction, or at least 3 log reduction, or at least 5 log reduction of the microorganism population of the equipment compared to the microorganism population before adding antimicrobial composition.

As a note, the ingredients described herein are not limited to certain composition. Any composition used in the present methods including CIP composition, enzyme composition, cleaning composition, and antimicrobial composition may selectively include one or more ingredients described anywhere in the present disclosure.

### Methods

In some aspects, the present disclosure relates to methods for cleaning industrial equipment. In particular, the present disclosure is related to methods for cleaning industrial equipment using a CIP process. The present methods may be used for one or more of the following purposes: removing soils, killing microorganisms, controlling a microorganism population, reducing biofilms, and removing contaminants from industrial equipment using CIP techniques; detecting or removing microorganisms or biofilm within the industrial equipment; analyzing, detecting, identifying, and determining biofilm and/or microorganisms of the industrial equipment to be cleaned; verifying elimination of biofilm and/or microorganisms from industrial equipment; reducing biofilm and/or microorganisms from industrial equipment; improving efficiency of the CIP process, providing a proof of cleanliness of the CIP process.

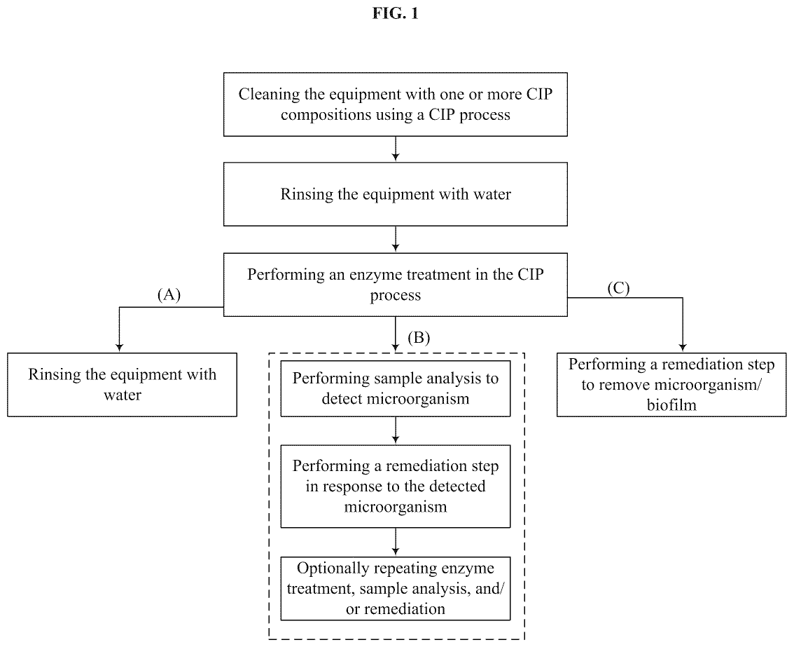

Now referring to FIG. 1-7, examples of the present methods and various aspects thereof will be illustrated and described in detail. FIG. 1 is a schematic illustration of the exemplary approaches adopted in the present disclosure for cleaning industrial equipment. Generally, the present methods include cleaning the equipment with one or more CIP compositions through the use of conventional CIP cleaning steps; rinsing the equipment with water to remove any CIP composition from the equipment; and performing an enzyme treatment by adding one or more enzyme compositions into the equipment absent any CIP composition. As discussed above, a distinct and separate enzyme treatment advantageously maximizes the efficiency of enzyme(s) in treating/detaching/dispersing the unremoved or residual or hidden microorganism or biofilm that still remain in the equipment after the conventional CIP cleaning. After enzyme treatment, at least three alternative approaches can be followed. In approach (A), the equipment upon effective enzyme treatment can be rinsed with water to remove the enzyme compositions and the dispersed microorganism/biofilm resulted from enzyme treatment. In approach (B), one or more samples of the fluid within the CIP process may be taken out and analyzed before, after, or before and after enzyme treatment to detect microorganisms within the CIP process. A remediation step may be performed in response to the detected microorganism or biofilm from the sample analysis. Optionally, repetition of the enzyme treatment, and/or the sample analysis, and/or additional remediation steps may be performed after the first remediation step to verify the elimination of microorganism/biofilm in the CIP process. In approach (C), a remediation step may be performed after the enzyme treatment to treat the dispersed microorganism in the fluid and further clean the equipment, without sample analysis.

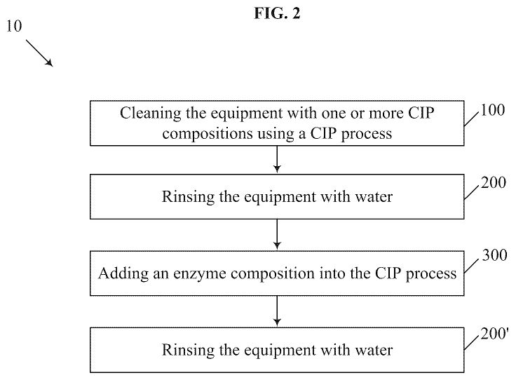

FIG. 2 illustrates a block diagram of an example method 10. In the illustrated example, the method 10 includes steps or operations 100, 200, 300, and 200′.

Operation 100 includes a CIP treatment. At 100, the industrial equipment is cleaned with one or more CIP compositions. The CIP compositions and the CIP process are described supra. As discussed above, the CIP treatment using CIP compositions could remove most soils, bulk soils, soil layer, soil particles, bulk mineral deposits, and bulk contaminants. Removal of soils typically exposes the interior or hidden biofilm layer that still remains attached to the surface of the industrial equipment. In some embodiments, the conventional CIP compositions could not remove all biofilms from the surface, especially those biofilms derived from certain microorganisms that have strong adhesion to the surface or fouling effect.

In some embodiments, the equipment is treated with the CIP composition at 100 for a period of time from about 5 minutes to about 120 minutes, or from about 15 minutes to about 90 minutes, or from about 30 minutes to about 60 minutes. In some embodiments, the CIP process is operated at an elevated temperature in the presence of the CIP composition or a use solution thereof from about 30° C. to about 100° C., or from about 40° C. to about 90° C., or from about 50° C. to about 70° C.

Operation 200 includes a rinse treatment. At 200, the industrial equipment is rinsed with water from a water resource, such as fresh water, tap water, deionized water, distilled water, soft water, or food-safe water. Water is allowed to flow or recirculate in the equipment at a speed from about 1 to about 10 feet/second and is then removed from the system. Rinsing the equipment with water after the operation 100 can remove the CIP composition used in the CIP treatment. In some embodiments, the industrial equipment is rinsed with water more than once to completely remove the residual CIP composition. In some embodiments, the rinsing step is performed at an elevated temperature from about 30° C. to about 100° C., or from about 40° C. to about 90° C., or from about 50° C. to about 70° C. In some embodiments, the rinsing step continues for a period of time from about 5 minutes to about 60 minutes, or from about 10 minutes to about 30 minutes, or from about 15 minutes to about 20 minutes.

Operation 300 includes an enzyme treatment. At 300, an enzyme composition is added to the CIP process. The enzyme composition may be added after the rinse treatment or operation 200. The enzyme composition may be pretreated before being added to the industrial equipment. Such pretreatment may include: diluting the enzyme composition to from a use solution; activating the enzyme(s) of the enzyme composition by adding enzyme activator or activity enhancer into the enzyme composition before use; heating the enzyme composition to a desired temperature, agitating/homogenizing the enzyme composition, etc. In some embodiments, the enzyme composition is dispensed from a source and added to the equipment, and a diluent such as water or solvent is added simultaneously to generate a use solution in situ. The formulation, ingredient, enzyme selection, enzyme concentration, and amount of the enzyme composition for operation 300 can vary depending on many factors including the size and complexity of the surface, the function of the equipment, the nature of the soil, microorganism, and biofilm, etc. Various aspects of the enzyme composition are described supra.

In some embodiments, operation 300 includes multiple enzyme treatments. For example, two or more enzyme treatments may be performed separately or successively, each enzyme treatment comprising using the same enzyme composition or different enzyme compositions targeting different microorganisms. In particular, each of the different enzyme compositions may have an enzyme or an ingredient or a pH that is not compatible with the other(s). A series of distinct and separate enzyme treatments may improve the total efficiency of biofilm/microorganism removal. In some embodiments, an optional step of rinsing the equipment with water may be added between any two successive enzyme treatment steps.

In some embodiments, the CIP process is operated at a temperature of about 30° C. to about 85° C., or from about 40° C. to about 70° C., or from about 50° C. to about 60° C., in the presence of the enzyme composition or a use solution thereof. In some embodiments, the enzyme composition added to the CIP process forms a use solution that has a pH value from about 2 to about 12, or from about 4 to about 10, or from about 7 to about 10. In some embodiments, the equipment is treated by each enzyme composition or a use solution thereof for a period of time from about 5 minutes to about 120 minutes, or from about 15 minutes to about 90 minutes, or from about 30 minutes to about 60 minutes.

Similar to operation 200, operation 200′ includes rinsing the equipment with water to remove the enzyme composition(s) used in operation 300.

In some embodiments, upon effective enzyme treatment in operation 300, operation 200′ will complete cleaning process without the need for additional steps.

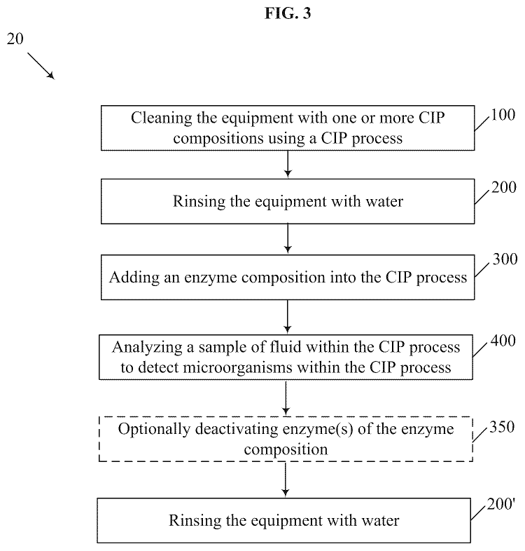

FIG. 3 illustrates a block diagram of another example method 20. In the illustrated example, the method 20 includes operations 100, 200, 300, 400, 350, and 200′. Operations 100, 200, 300, and 200′ have been described according to the method 10 and will not be repeated here.

Operation 400 includes a biofilm/microorganism analysis step. At 400, a sample of fluid within the CIP process is obtained and analyzed to detect microorganisms within the CIP process. The sample can be taken out from the fluid within the CIP process before, after, or before and after the addition of the enzyme composition. The analysis of the sample taken out from the fluid before the addition of the enzyme composition can provide information of background level of planktonic or free-floating microorganisms present in rinse water. The analysis of the sample taken out from the fluid after the addition of the enzyme composition can provide information of the type of biofilm microorganisms that are removed by the enzyme treatment. In some embodiments, operation 400 further includes comparing the analytical results from samples taken out from the fluid both before the addition of the enzyme composition and samples after the addition of the enzyme composition. The sample analysis (before enzyme treatment, after enzyme treatment, or the comparison of the data before and after enzyme treatment) can provide information about the microorganism or biofilm that is useful to for the selection of the enzyme for the enzyme composition or the subsequent microorganism/biofilm remediation step. Such information may also be useful to guide CIP cleaning of industrial equipment of the similar type of function.

In some embodiments, operation 400 employs one or more microbial detection methods or techniques. Examples of the microbial detection methods include but are not limited to: in-plate culture identification, bioluminescence assay, ATP photometry, PCR based rapid detection technique and/or a biosensor, immunoassay, bioburden test; microorganism/biofilm cell staining and subsequent optical quantification technique, a microorganism/biofilm extracellular polymeric substances (EPS) staining and subsequent optical quantification technique, HPLC, flow cytometry, microscopy, FISH, oxygen consumption measurement, CO₂ production measurement, measurement production of metabolites, or any combinations thereof.

### In-Plate Culture Identification

In some embodiments, operation 400 includes a step of conducting a bacteriological analysis of the sample fluid within the CIP process. Such analysis measures, estimates, quantifies, or determines the number of bacteria present and, if needed, the identity of the bacteria or its metabolic state. Exemplary methods include: multiple tube method, luminescence assay, plate count, membrane filtration, and pour plates.

In some embodiments, operation 400 comprises using an in-plate culture identification method to analyze the sample fluid. In some embodiments, the culture identification method includes cultivating the sample on a culture medium in a plate and performing a standard plate count for one or more microorganism including but not limited to bacteria, yeast and mold, psychrophiles, psychrotrophs, mesopiles, thermophiles, aerobes, anaerobes, facultative anaerobes, bacteria spore former, spoilage microorganism, pathogens, specific biofilm indicator microorganism, or any combinations thereof. Methods of cell culture and plate count are generally known in the art.

Examples of the biofilm microorganisms include both initial biofilm-formers and subsequent biofilm adaptors, archaebacteria, aerobic bacteria, anaerobic bacteria, facultative anaerobic bacteria, hydrocarbon oxidizing organisms, iron bacteria, sessile bacteria, strict anaerobe, plankton, zooplankton, phytoplankton, biofilm bacteria, surface attached (sessile) bacteria, algae, protozoa, fungi, copepods, planktonic bacteria, thermophilic bacteria, sulfur-oxidizing bacteria, sulfate-reducing bacteria, iron bacteria.

Bacterial species that may be detected from the fluid sample analysis include diverse taxa/species from Bacteroidetes, Proteobacteria, Firmicutes, and other bacteria phyla, Achromobacter, Acidobacteria, Acinetobacter, Aeromonas, Agrobacterium, Alcaligenes faecalis, Alteromonadaceae (such as Pelagibacter), Bacillaceae bacterium, such as Bacillus acidogenesis, Bacillus cereu, Bacillus cogaulans, Bacillus flavothermus, Bacillus licheniformis, Bacillus macrolides, Bacillus megaterium, Bacillus sphaericus, Bacillus sporothermodurans, Bacillus subtilis, Bacteriodetes, Brachybacterium species, such as Brachybacterium paraconglomeratum, Brevibacterium species, such as Brevibacterium casei, Brevundimonas species, such as Brevundimonas diminuta, Burkholderia species, such as Burkhokderia vienamiensis, Burkholderia cepacia, Burkholderiaceae (such as Burkholderia and Comamonas), Campylobacter jejuni, Cellulomonas cellasea, Cellulomonas gelida, Clostridium species, such as Clostridium botulinum and Clostridium perfringens, Comamonadaceae, Cytophaga arvensicola, Deinococcus grandis, Delftia species, such as Delftia acidovorans, Dietzia species, Escherichia coli, Enterobacteriaceae, Flavobacterium spiritivorum, Gluconacetobacter, Geobacillus species, Geobacillus stearothermophilis, Halobacterium salinarum, Hippea species, such as Hippea maritime, Klebsiella, Lactobacillus, Lactococcus, Leptotrichia species, Listeria monocytogenes, Marinobacter, Methanocalculus pumilus, Methanocaldococcus interns, Methanoculleus thermophilitcus, Methanomethylovorans victoriae, Methanosarcina barkeri, Methanosarcina mazei, Methanothermobacter thermautotrophicus, Methanothrix soehngenii, Methylobaceterium species, such as Methylobaceterium rhodinum, Microbacterium, Micrococcus, Moraxellaceae, Ochrobactrum species, such as Ochrobactrum anthropi, Ochrobactrum grignonense, and Ochrobactrum tritici, Oxalobacteraceae, Panibacillus, Peptostreptococcus species, Polphyromonas species, Propionibacterium acnes, Proteobacteria, Pseudomonadaceae, such as Pseudomonas flourescens, Pseudomonas aeruginosa and Pseudomonas putida, Pyrodictium occultum, Ralstonia, Salmonella, a bacteria from SARI1 clade, a lineage of bacteria from the Alphaproteobacteria class, Shigella species, Slenotrophomonas maltophilia, Sphigomonas, Staphylococcus species, such as Staphylococcus aureus and Staphylococcus lentus, Stenotrophomonas species, such as Stenotrophomonas maltophilia, Streptococcus A, Tissierella species, Veillonellaceae species, such as Pelosinus, Vibrio species, such as Vibrio cholerae, Vibrio parahaemolyticus, and Vibrio vulnificus, Xanthomonadaceae, and Yersina.

### ATP Photometry and Bioluminescence Assay

In some embodiments, operation 400 includes measuring microbial activity of the sample taken out from the fluid within the CIP process. One way of detecting microorganisms depends on measuring microbial activity. Microbial activity can be measured using adenosine triphosphate (ATP) concentrations as an indicator of activity. ATP measurements have been used for detecting microorganisms in various industries. ATP is used by cells as a source of energy and is an indicator of metabolic activity. Microbial activity can also be measured using metabolic dyes, including redox dyes (e.g. resazurin and 2-(p-iodophenyl)-3-(p-nitrophenyl) 5-phenyltetrazoliurn chloride (INT)), fluorescent redox dyes (e.g. 5-cyano-2, 3-ditolyl tetrazolium chloride (CTC)) and indicators of enzymatic activity (e.g. carboxyfluorescien diacetate).

In some embodiments, operation 400 includes using an ATP photometry method to analyze the sample fluid. In such embodiments, the ATP photometry method is used to measure the ATP level of a sample using bioluminescence assay. Methods related to ATP photometry are generally known in the art. For example, ATP can be measured by adding luciferase and luciferin to the sample fluid and measuring light emissions in relative light units (RLUs). ATP can also be measured by HPLC. In other embodiments, the microbial activity of the fluid sample is detected by metabolic dyes as described supra.

In an example of ATP photometry utilizing bioluminescence, luciferase and luciferin from fireflies are mixed with a fluid sample of the CIP process, a detergent is used to release microbial (intracellular) ATP, and a cation, such as magnesium, in the presence of oxygen. If microbial ATP is present, it will cause a reaction between luciferase (the substrate) and luciferin (the catalyst) in an oxidation reaction which produces light. Light emissions are detected with a luminometer and reported in relative light units (RLUs). The amount of light produced is proportional to the metabolic activity of microbial organisms present, but does not indicate the number of organisms present. The luciferase/luciferin reaction is well known in the art, and there are commercial sources for the necessary reagents as well as protocols for their use. For example, several luciferase/luciferin reagents along with luciferase are available in commercial kits from, for example, Promega Corp. (Madison, Wis.) and LuminUltra (Fredericton, New Brunswick). Commercially available luciferases include firefly luciferase (Photinus pyralis, “Ppy luciferase”). Purified beetle luciferin is also commercially available from Promega.

In some embodiments, ATP levels are monitored over time, by taking ATP readings at specified time intervals. New samples are taken at each time interval and combined with ATP reagents to produce each luminescence reading. ATP measurements may be taken every 2 hours, every 1 hour, every 30 minutes, every 15 minutes, or every 5 minutes. Alternatively, HPLC may be utilized to measure ATP levels in the sample. HPLC measurements may also be taken at least every hour to monitor ATP levels. The same procedure may be conducted using dyes to detect microbial activity based on redox changes or metabolic activity. Samples can be examined for visual evidence of a color change, spectrophotometrically or by measuring fluorescence.

In some embodiments, the ATP photometry method described herein includes: measuring an initial ATP concentration in a fluid sample, extracting Adenosine monophosphate (AMP) and/or adenosine diphosphate (ADP) from the fluid sample; converting the AMP and/or the ADP to ATP using one or more enzymes; and measuring the level of the converted ATP. Once a final quantity of ATP is measured, an increase in ATP concentration from the initial ATP measurement to the converted ATP measurement indicates the presence of bacteria or spores in the fluid sample. In some aspects, AMP and ADP are extracted from the sample with solvent, acid, heat, or surfactant. In embodiments, an initial ATP measurement is not taken and the samples are treated with heat at temperatures of 65° C. or higher for 1 to 10 minutes in order to kill any vegetative bacteria present. In this method, any ATP that is converted from AMP or ADP would be attributed to spores that survive the heat treatment. In some embodiments, the AMP and ADP are converted to ATP by treating the sample with pyruvate kinase and phosphoenolpyruvate to convert ADP to ATP, treating the sample with myokinase to convert ATP and AMP to ADP, and treating the sample with pyruvate kinase and phosphoenolpyruvate to convert ADP to ATP. In some embodiments, the adenine nucleotide is converted to ATP with pyruvate kinase, myokinase, and phosphoenolpyruvate within about 10 minutes to 24 hours.

### DNA-Based Analysis

In some embodiments, operation 400 includes a DNA-based analysis of the fluid sample within the CIP process. In some embodiments, the DNA-based analysis utilizes a PCR-based rapid detection technique and/or a biosensor. PCR-based detection methods are generally known in the art. In at least one embodiment, the DNA-based analysis involves the use of PCR primers to detect the presence or absence of microorganisms. U.S. Pat. No. 5,928,875 describes the use of PCR primers to detect the presence or absence of spore forming bacteria. In at least one embodiment the primer is targeted towards a part of a DNA strand which is highly conserved among a group of microorganisms. As a result, detecting the presence of that particular part of DNA is definitive proof of the presence a specific microorganism. PCR analysis is of particular use in analyzing samples with a low concentration of microorganisms or where there is a low concentration of viable microorganisms. ATP measurements require either a certain amount of microorganisms or a certain amount of viable microorganisms.

In at least one embodiment the PCR analysis is a qPCR analysis as described in Trade Brochure qPCR guide, prefaced by Jo Vandesompele, (as downloaded from website http://www.eurogentec.com/file-browser.html on Jan. 19, 2012). In at least one embodiment the method is a quantitative qPCR analysis. In at least one embodiment the method is a qualitative qPCR analysis.

As illustrated in at least one embodiment, once DNA is extracted from the sample, using any of the DNA extraction kits available commercially, it can be analyzed in real-time using a PCR approach such as a quantitative PCR approach. Quantitative PCR utilizes the same methodology as PCR, but it includes a real-time quantitative component. In this technique, primers are used to target a DNA sequence of interest based on the identity of the organism or function of a specific gene. Some form of detection such as fluorescence may be used to detect the resulting DNA or “DNA amplicon.” The change in fluorescence is directly proportional to the change in the quantity of target DNA. The number of cycles required to reach the predetermined fluorescence threshold is compared to a standard that corresponds to the specific DNA target. A standard is typically the target gene that is pure and of known quantity at concentrations that span several logs. The number of copies of target DNA present in the sample is calculated using the standard curve. The copy number per sample is then used to determine the number of cells per sample.

In at least one embodiment more than one primer is used to identify microorganisms that have more than one uniquely recognizable nucleotide sequence. In at least one embodiment the PCR analysis is used to detect genome sequences associated with enzymes unique to or nearly unique to specific microorganisms.

More examples of methods for detecting and identifying microorganism using DNA-based analysis can be found in U.S. Pat. No. 8,613,837 and U.S. Pat. Pub. No. US 2016/0304931, which are incorporated by references herein in their entirety.

### Additional Analysis Options

In some embodiments, operation 400 includes staining microorganism/biofilm cells of the fluid sample and subsequently measuring the number of the stained cells by an optical quantification technique. Cell staining and optional quantification techniques are generally known in the art. Typically, the fluid sample of the CIP process is contacted with a staining solution to stain the cells or extracellular polymeric substances (EPS) of microorganisms from the biofilm for a certain amount of time, then the number of stained cells or the quantity of the EPS are measured by an optical technique. Common optional techniques include microscopy, flow cytometry, fluorescence in situ hybridization (FISH), etc.

In some embodiments, operation 400 includes one or more steps selected from the group of measuring oxygen consumption, measuring CO₂ production, measuring production of metabolites, or any combinations thereof.

In some embodiments, microbial activity of the fluid sample within the CIP process can be indirectly measured by monitoring the consumption of dissolved oxygen (DO) because dissolved oxygen consumption is directly related to the amount of ATP that a cell is producing and the amount of ATP that a cell produces can be correlated with the level of microbial activity in said samples. Microorganism or biofilm amount or activity can be calculated by the difference in DO measurements taken for the samples before the addition of enzyme composition and after the addition of enzyme composition.

As discussed above, operation 400 includes identifying or determining and/or quantifying the detected microorganisms such as bacteria or spores. If no microorganism is detected or the detected microorganism is below a threshold level or within an acceptable range, an indication of microorganism/biofilm elimination can be verified, and no subsequent remedial step will be needed.

In some embodiments, the method 20 may further include an optional step 350 of deactivating or inhibiting enzyme(s) of the enzyme composition used in operation 300. At 350, deactivation or inhibition of enzyme(s) may be achieved by chemical intervention such as using an enzyme inhibitor, a cleaning composition, an antimicrobial composition, an acid, etc. Other means to deactivate enzyme(s) include changing the pH of the fluid within the CIP process; elevating the temperature in the CIP process; diluting the fluid with water, etc. In some embodiments, rinsing the equipment with water at 200′ may effectively remove enzyme(s) of the enzyme composition.

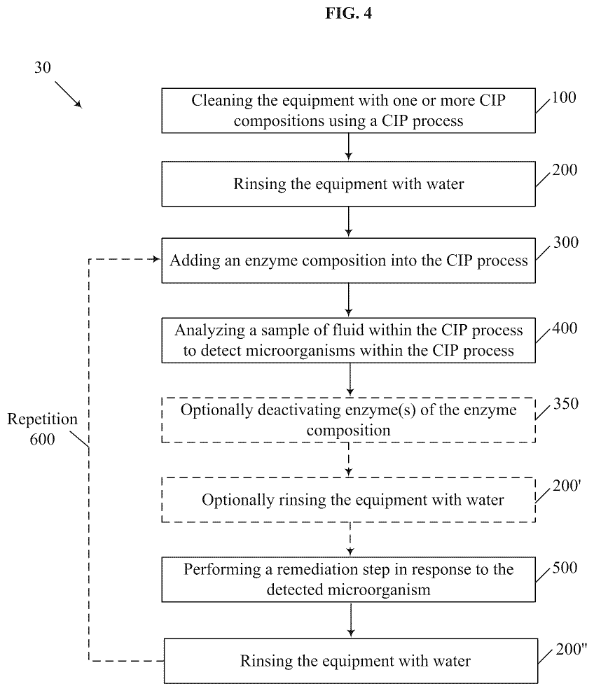

FIG. 4 illustrates a block diagram of yet another example method 30. In the illustrated example, the method 30 includes operations 100, 200, 300, 400, 500, 200″. The method 30 may optionally include operations 350, 200′, and 600. Operations 100, 200, 300, 350, 400, and 200′ have been described according to the methods 10 and 20.

Operation 500 includes performing a remediation step 500 after operation 400. In particular, when certain biofilm or microorganism has been detected and identified at 400, the microorganism/biofilm remediation step 500 is preferred to further clean the equipment and eradicate the detected biofilm or microorganism that still remain in the equipment.

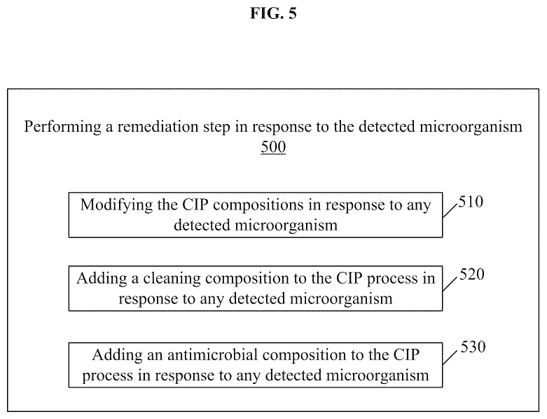

FIG. 5 illustrates a block diagram of one example operation 500. In the illustrated example, operation 500 includes one or more of the operations 510, 520, and 530. Operation 510 includes an additional cleaning treatment by modifying the CIP compositions in response to any microorganism detected in operation 400. Operation 520 includes an additional cleaning treatment by adding a cleaning composition to the CIP process. Operation 530 includes an additional cleaning treatment by adding an antimicrobial composition to the CIP process.

### Gas-Enhanced Cleaning

In one particular embodiment, a gas-enhanced treatment is used at 520 to facilitate removal the microorganism/biofilm. The gas-enhanced cleaning technique is found to be effective in treating hard soils and dissociating biofilm matrix that is difficult to remove. As an example, step 520 may include adding a gas-releasing solution to the CIP process. The gas-generating use solution may be present in either a pretreatment solution or an override use solutions. The gas generating use solution is applied to the equipment surface for an amount of time sufficient to allow the solution to penetrate the soil. A gas, such as O₂ or CO₂, may be generated either spontaneously or upon trigger by an override solution, in an amount sufficient to provide a disruption effect which substantially removes the microorganisms or biofilm from the surface by loosening or breaking up the biofilm or microorganisms from the surface. The loosened biofilm or microorganisms can then be easily washed away.

Examples of oxygen- and carbon dioxide-releasing methods are found in in U.S Patent Publication No. 2009/0200234 and U.S. Pat. No. 10,099,264, which are incorporated herein by reference.

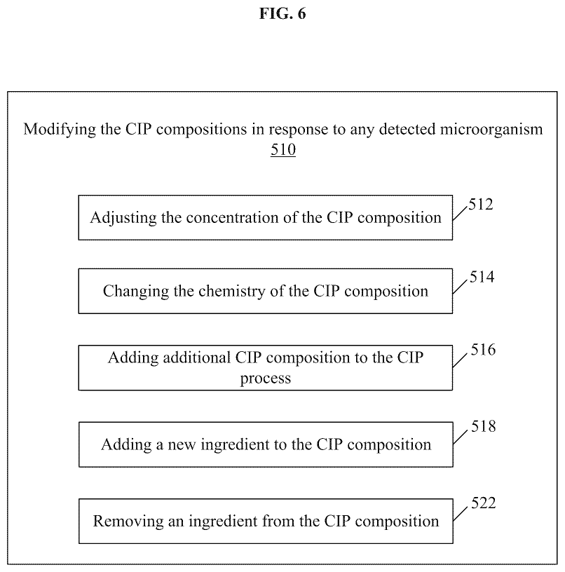

FIG. 6 illustrates a block diagram of one example operation 510. In the illustrated example, operation 510 include one or more of the following operations: 512, 514, 516, 518, and 522. At 512, the concentration of the CIP composition is adjusted in response to the detected microorganism in the enzyme treatment of operation 400. In some embodiments, the total amount of CIP composition and/or the diluent is adjusted to control the concentration of active ingredients in the use solution derived from the CIP compositions.

At 514, the chemistry and/or operating condition of the CIP composition is changed in response to the detected microorganism in the enzyme treatment of operation 400. The chemistry of the CIP compositions broadly includes content or ratio of the ingredients thereof, pH, ion strength, viscosity, operating temperature, duration of treatment time, pressure, gas generation, scrubbing, etc.

At 516, additional CIP composition is added to the CIP process in response to the detected microorganism in the enzyme treatment of operation 400. The additional CIP compositions may be the same as the CIP composition used prior to the enzyme treatment or different in chemistry, formulation, and concentration.

At 518, one or more new ingredients is added to the CIP composition in response to the detected microorganism in the enzyme treatment of operation 400. For example, the new ingredient may be an antimicrobial agent or cleaning composition that has not been used in the CIP compositions prior to the enzyme treatment of 400. The new ingredient may be selected to specifically target against the detected microorganism.

At 522, one or more ingredients may be removed from the CIP composition in response to the detected microorganism in the enzyme treatment of operation 400. For example, an acid or base may be removed from the CIP composition to adjust the CIP composition to a desired pH range suitable for removing the detected microorganism.

In some embodiments, operation 500 can further include one or more cleaning treatments of the equipment using the modified CIP compositions of operation 510. Different compositions modified in operation 510 may be used in a single treatment or separate treatments of operation 500. For example, operation 500 may include cleaning the equipment using a modified acid CIP composition followed by a modified alkaline CIP composition to remove the microorganisms identified and detected from operation 400.

In some embodiments, the microorganism/biofilm remediation step is operated at a temperature of about 30° C. to about 85° C., or from about 40° C. to about 70° C., or from about 50° C. to about 60° C. In some embodiments, the microorganism/biofilm remediation step continues for a period of time from about 5 minutes to about 120 minutes, or from about 15 minutes to about 90 minutes, or from about 30 minutes to about 60 minutes.

In some embodiments, the remediation step 500 through the use of a cleaning composition or an antimicrobial composition may deactivate enzymes of the enzyme composition used in 300. In such cases, a separate enzyme deactivation step (such as 350) may not be needed.

In some embodiments, operation 200 may be performed to rinse the equipment with water to remove the enzyme composition before the remediation step.

Operation 200″, similar to operations 200 and 200′, includes rinsing the equipment with water to remove the chemicals used in the remediation step 500.

In some embodiments, the method 30 further includes a repetition step 600. One or more or all of the operations 300, 400, 350, 200′, 500, and 200″ may be repeated at 600. In one example, a second enzyme treatment is performed by adding a second enzyme composition into the CIP process after 200″. After the second enzyme treatment a sample of the fluid within the CIP process is taken out and analyzed to detected remaining microorganism or biofilm. A second remediation step may be followed in response to the detected microorganism/biofilm to further improve the cleaning efficiency of the entire process. The repetition step 600 may continue until there is an indication of acceptable cleanliness of the equipment.

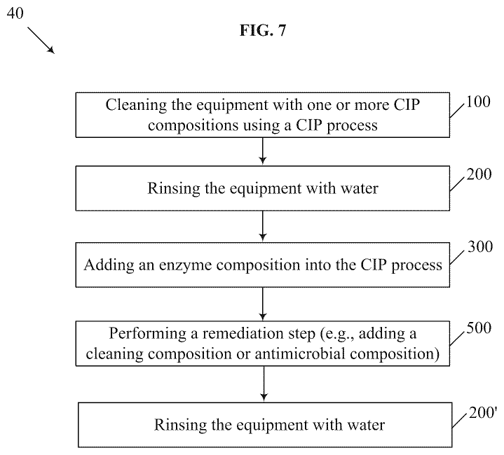

FIG. 7 illustrates a block diagram of another example method 40. In the illustrated example, the method 40 includes operations 100, 200, 300, 500, and 200′, which are described according to methods 10, 20, 30, and will not be repeated here. It is noted the remediation step at 500 may be performed subsequently after operation 300 without a separation step of enzyme deactivation. In one example, a cleaning composition or an antimicrobial composition can be added at 500. The cleaning composition and/or the antimicrobial composition may include one or more antimicrobial agent that specifically targets against the remaining microorganism and deactivates the enzyme(s) used at 300. In some embodiments, the cleaning solution is an alkaline cleaning solution described supra. The cleaning composition may also be an additional enzyme composition used in operation 300 or a new enzyme composition having a particular enzyme that targets against the remaining microorganism. In one particular embodiment, a cleaning booster or a bubble enhanced cleaning solution described supra is used at 500 in combination with the cleaning composition or antimicrobial composition to improve the cleaning efficiency.

In some embodiments, the method 30 includes no sample analysis step between operations 300 and 500, comparing with the method 10. In such embodiments, the enzyme treatment of operation 300 may destroy the biofilm matrix or network, soften or loosen the biofilm or microorganisms, detach or partially detach the biofilm or microorganisms from the surface, and suspend/stabilize the detached biofilm or microorganisms in the fluid. The subsequent remediation step 500 may be performed directly to clean off the loosened or softened or detached biofilm or suspended microorganisms/biofilm without necessity to analyze the microorganism of the biofilm, especially when the type of the biofilm or microorganism is ascertained based on prior knowledge.

In some embodiments, the methods according to the present disclosure result in at least 1 log reduction, or at least 2 log reduction, or at least 3 log reduction, or at least 5 log reduction of the microorganism population of the equipment compared to the microorganism population before adding the enzyme composition.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

### EXAMPLES

Embodiments of the present disclosure are further defined in the following non-limiting examples. It should be understood that these examples, while indicating certain embodiments of the disclosure, are given by way of illustration only. From the above discussion and these examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

Tables 1 and 2 show the commercial enzyme compositions and cleaning composition used in the examples.

TABLE 1
Commercial enzyme compositions

| Name | Source | Description |
|---|---|---|
| AD Anios EZ | Laboratoires Anios | An enzymatic complex containing |
| | (a subsidiary of | protease, lipase, and amylase. |
| | Ecolab Inc., MN) | AD Anios EZ may be used in |
| | | combination with an enzyme |
| | | buffering product Ultrasil 69 |
| | | from Ecolab. |
| Soluscope EZ | Soluscope (a | An tri-enzyme detergent complex |
| | subsidiary of | containing protease, lipase, and |
| | Ecolab Inc., MN) | amylase. |

TABLE 2
Commercial cleaning compositions

| Name | Source | Description |
|---|---|---|
| Exelerate HS-I | Ecolab Inc. (MN) | A cleaning booster of active oxygen. |
| Exelerate CA | Ecolab Inc. (MN) | A heavy duty alkaline cleaner. |
| AC-110 | Ecolab Inc. (MN) | An alkaline cleaner |
| DAC-110 | Ecolab Inc. (MN) | An alkaline cleaner |
| Vortexx | Ecolab Inc. (MN) | A mixed peracid based disinfectant/ |
| | | antimicrobial composition |

### Example 1—Microorganism Cleaning by Enzyme Treatment and Cleaning Treatment Using Center for Disease Control (CDC) Biofilm Reactor

ASTM E2562-17 was used to grow reproducible Pseudomonas aeruginosa biofilm on 304 stainless steel coupons under high shear and continuous flow using a CDC biofilm reactor (obtained from BioSurface Technologies Co., Montana). After 48 hours, the growth phase was complete, the biofilm-covered coupons and coupon holders were rinsed and transferred to a new CDC biofilm reactor. 500 ml of an enzyme composition and cleaning composition were added sequentially to the reactor and the biofilm coupons were treated at the temperature and contact time specified in the procedures shown in Table 3 under 120 rpm.

TABLE 3
Procedures and analytical results for microorganism treatment of stainless
steel coupons using Soluscope EZ, Exelerate HS-I, and AC-110.

| | | | | Biofilm Analysis Results | |
|---|---|---|---|---|---|
| | Biofilm Treatment Procedure | | | Biofilm remaining | Biofilm dispersed |
| | | Temperature | Contact | on 304SS coupon | into cleaning solution |
| Steps | Products | (° C.) | (min) | (CFU/coupon) | (CFU/ml) |
| 0 | Biofilm baseline | | | 3.6E+08 | n/a |
| 1 | Sterile water rinse | | | | |
| 2 | 0.5% Soluscope EZ | 50° C. | 30 | 1.1E+07 | 38.1E+06 |
| 3.1 | 0.5% Exelerate HS-I | 85° C. | 15 | | |
| 3.2 | 2% AC-110 | 85° C. | 45 | 2.3E+03 | <1.0E+00 |

| | |
|---|---|
| 4 | Sterile water rinse |

After the desired contact time, the treated coupons were removed from the CDC reactor, and the biofilm remaining on the coupons was segregated and analyzed by cell culture plating for viable cell enumeration, according to ASTM E2871-19. Biofilm dispersed into cleaning solution was also quantified by plating a sample of the respective cleaning composition.

The enzyme treatment alone resulted in a 1.5 log reduction of the biofilm on the coupons. At the same time, a significant increase of viable cells was detected in enzyme cleaning solution. These results show that enzyme treatment could effectively disperse biofilm from hard surfaces (e.g., surface of stainless steel coupon) into the solution. The enzymatic compositions and treatment provided more than 90% (or >1 log) biofilm recovery and could serve as an effective sampling method for interior biofilm in closed systems or other difficult to access surfaces. Dispersed biofilm cells could further be analyzed by cell plate count or microbial detection methods to quantify the amount of microorganism.

The subsequent cleaning treatment resulted in more than a 5 log reduction in the biofilm on the coupons compared to the baseline. It is noted that once dispersed in solution and no longer protected by the biofilm matrix, the biofilm microorganisms could not survive the harsh condition of alkaline environment, thus viable cells could not be detected in samples taken from the solution containing alkaline cleaner.

### Example 2—Microorganism Treatment by Enzyme/Cleaner/Antimicrobial and Biofilm Analysis in Pilot UHT

Biofilm was developed on interior pipes and surfaces of pilot UHT equipment (PT-20 Mini UHT by Powerpoint International Ltd.) from bacteria naturally present in milk. After a pasteurization cycle, milk was discharged, followed by a continuous water rinse to flush out suspended bacteria, and then UHT was soaked in a final rinse water at ambient temperatures for 3 days to allow biofilm to mature.

The UHT equipment containing developed biofilm was sequentially treated with an enzyme composition, a cleaning composition, and an antimicrobial agent. Tables 4 and 5 provide the procedures of two separate biofilm treatment experiments.

TABLE 4
Procedures and analytical results for microorganism treatment
in pilot UHT using Soluscope EZ, Exelerate CA, and Vortexx.

| | Biofilm Treatment Procedure | | | Biofilm Analysis Results | |
|---|---|---|---|---|---|
| | | Temperature | Contact | ATP Total | Viable Plate Count |
| Steps | Products | (° C.) | (min) | (RLU) | (CFU/ml) |
| 1 | Water rinse 5 min | | | | |
| 2 | 0.5% Soluscope EZ | 50° C. | 0 | 7 | 2.26E+03 |
| | | | 30 | 20 | 1.12E+05 |
| | | | 60 | 39 | 1.48E+05 |
| | | | 120 | 46 | 2.02E+05 |
| 3 | 3% Exelerate CA | 90° C. | 30 | | |
| 4 | Water rinse 5 min | | | | |
| 5 | 0.5% Soluscope EZ | 50° C. | 0 | 0 | 0 |
| | | | 30 | 0 | 2 |
| | | | 60 | 0 | 7 |
| | | | 120 | 0 | 4 |
| 6 | 0.26% Vortexx | 60° C. | 10 | | |

| | |
|---|---|
| 7 | Water rinse 5 min |

In one process according to Table 4, the UHT equipment was rinsed with water at step 1, and then was treated with an enzyme composition (0.5% Soluscope EZ) at step 2. Subsequently, the UHT equipment was treated by an alkaline cleaner (3% Exelerate CA) for 30 mins at step 3. The UHT equipment was rinsed with water at step 4 to remove the alkaline cleaner. To evaluate the effectiveness of microorganism removal, a second enzyme treatment with the same enzyme composition (0.5% Soluscope EZ) was performed at step 5, followed by a mixed peracid based disinfectant/antimicrobial composition (0.26% Vortexx) at step 6. The UHT equipment was finally rinsed with water at step 7. A similar process was performed according to the procedures of Table 5, using a different enzyme composition.

TABLE 5
Procedures and analytical results for microorganism treatment in
pilot UHT using Ultrasil/Anios EZ, Exelerate CA, and Vortexx.

| | Biofilm Treatment Procedure | | | Biofilm Analysis Results | |
|---|---|---|---|---|---|
| | | Temp | Contact time | ATP | |
| Steps | Product | (° C.) | (min) | total | CFU/ml |
| 1 | Water rinse 5 min | | | | |
| 2 | 0.1% Ultrasil 69 + | 50° C. | 0 | 30 | 3.00E+04 |
| | 0.2% AD | | 10 | 43 | 4.30E+04 |
| | Anios EZ | | 20 | 35 | 4.30E+04 |
| | | | 60 | 33 | 7.60E+04 |
| 3 | 3% Exelerate CA | 90° C. | 30 | | |
| 4 | Water rinse 5 min | | | | |
| 5 | 0.1% Ultrasil 69 + | 50° C. | 0 | 7 | 2 |
| | 0.2% AD | | 10 | 0 | 1 |
| | Anios EZ | | 20 | 0 | 4 |
| | | | 60 | 4 | 2 |
| 6 | 0.26% Vortexx | 60° C. | 10 | | |

| | |
|---|---|
| 7 | Water rinse 5 min |

A sample of enzyme cleaning solution was analyzed to verify biofilm presence via ATP test by Hygiena AquaSnap. Total cell counts were determined using cell plating. Note that ATP is preferred to analyze samples having relatively high amount of bacteria because ATP can usually provide quick result in less than 1 minute but its detection limit of ATP for bacteria cells is about 1000 CFU/ml.

In each of the processes as shown in Tables 4 and 5 respectively, during the first enzyme cleaning treatment (step 2), an increasing amount of ATP content and plate counts were observed over time in both Soluscope EZ (Table 4) and AD Anios EZ (Table 5) enzyme solutions, indicating that more microbial cells were released from UHT surfaces into solution as a result of enzyme treatment. The subsequent addition of Exelerate CA cleaning composition to the UHT equipment allowed for further removal, loosening, or weakening of the biofilm/microorganism. After the addition of Exelerate CA cleaning composition, a second enzyme cleaning (step 5) was carried out to quantify remaining biofilm or to verify biofilm removal. As shown in Tables 4 and 5, a significant decrease of the total ATP to low or no detectable levels were observed, and cell counts were also found to significantly decrease to a level of the rinse water background <10 CFU/ml. Comparing to the initial biofilm levels in step 2, a substantial improvement (with 4-5 log) of biofilm removal efficiency was achieved by combination of enzyme treatment and subsequent alkaline cleaning composition, resulting in little or no biofilm remaining inside the UHT equipment after the process.

### Example 3—Microorganism/Biofilm Treatment by Enzyme/Cleaner/Antimicrobial Agent and Biofilm Analysis in a Dairy Processing Equipment

Biofilm cleaning processes in pilot or industrial scale employing the methods of the present disclosure were also conducted. In this Example, a UHT milk manufacturing line was suspected of biofilm contamination by a food producer who experienced random microbial quality defects in the final product but had previously screened out all other sources of contamination. A CIP program was designed to solve the residual biofilm problem arising from the conventional CIP process using the following steps: The UHT milk line was first treated by an alkaline cleaning step, an acid cleaning step, and a hot water sanitization step; followed by a water rinse at step 2. The background microbial level in the water was determined at step 3. After a water rinse at step 4, the UHT milk line was treated by an enzyme composition (0.1% Ultrasil 69+0.2% AD Anios EZ) at step 5. Biofilm sampling and detection by enzyme treatment was also performed at step 5 to quantify the residual microorganism/biofilm level after enzyme treatment. Subsequent treatment of the UHT milk line using cleaning compositions was performed at steps 6.1 (using 0.5% Exelerate HS—I) and step 6.2 (3.5% DAC-110). After a water rinse at step 7, the UHT milk line was further treated using a mixed peracid based antimicrobial composition (0.5% Vortexx) at step 8. Following a water rinse at step 9, a second enzyme treatment was performed at step 10 using the same enzyme composition of step 5. Biofilm sampling and detection was performed after step 10 to verify biofilm removal. A water rinse was performed to remove the enzyme composition at step 11. As precaution, a final treatment using an alkaline CIP composition, an acid CIP composition, and a hot water sanitization step was performed at step 12 before the UHT milk line returned to production. Detailed procedures of the CIP program are provided in Table 6.

TABLE 6
Procedures and analytical results for microorganism treatment
in the UHT milk manufacturing line according to Example 3.

| | | | | | Biofilm Analysis Results | |
|---|---|---|---|---|---|---|
| | Biofilm Treatment Procedure | | | | Total Bacteria, | Thermophilic Spores, |
| | | Temp | | Contact time | average of 5 samples | average of 5 samples |
| Steps | Product | (° C.) | | (min) | (CFU/100 ml) | (CFU/100 ml) |
| 1 | Alkaline and Acid CIP cleaning | | | | | |
| 2 | Water rinse 15 min | | | | | |
| 3 | Water | 45~50° | C. | 60 | 2 | 3 |
| 4 | Water rinse 15 min | | | | | |
| 5 | 0.1% Ultrasil 69 + | 45~50° | C. | 60 | 33 | 0 |
| | 0.2% AD Anios EZ | | | | | |
| 6.1 | 0.5% Exelerate HS-I | 60~70° | C. | 15 | | |
| 6.2 | 3.5% DAC-110 | 80~85° | C. | 30 | | |
| 7 | Water rinse 15 min | | | | | |
| 8 | 0.5% Vortexx | 50~60° | C. | 15 | | |
| 9 | Water rinse 15 min | | | | | |
| 10 | 0.1% Ultrasil 69 + | 50° | C. | 60 | 5 | 6 |
| | 0.2% AD Anios EZ | | | | | |

| | |
|---|---|
| 11 | Water rinse 15 min |
| 12 | Alkaline and Acid CIP cleaning |

Samples taken from the liquid within the CIP process were analyzed to verify biofilm presence in the liquid via plating total bacteria and thermophilic spores using a standard plate method. When a larger sample size (example 100 ml) was needed to verify biofilm elimination, membrane filtration was used to capture all cells onto a membrane before plating.

Table 6 shows that the microbial analysis of a sample of recirculating water from step 3 had a very low background level of bacteria in the water. In comparison, the enzyme treatment at step 4 suspended hidden biofilm from equipment surface into the liquid, as evidenced by the significant (10×) increase of total bacterial counts in the liquid sample (33 CFU/100 ml). It is noted that the original amount of bacterial on the equipment surface before the first enzyme treatment (step 5) was estimated to be about 100 to about 1,000 times more concentrated than the bacterial count in the liquid sample, which was sufficient to cause quality issues. After biofilm elimination at steps 6.1 and 6.2 and 8, a second enzyme treatment at step 10 was performed to verify the biofilm removal. The total bacterial count was found to be at a significantly low level (5 CFU/100 ml), which was similar to the background water (2 CFU/100 ml), indicating an effective biofilm remediation/elimination from the UHT equipment.

The above specification, examples and data provide a complete description of the manufacture and use of the composition of the disclosure. Since many embodiments of the disclosure can be made without departing from the spirit and scope of the disclosure, the disclosure resides in the claims hereinafter appended.

1. A method for detecting or removing a microorganism or biofilm within industrial equipment, the method comprising:
(1) cleaning the equipment with one or more CIP compositions using a CIP process;
(2) rinsing the equipment with water;
(3) adding an enzyme composition into the CIP process; and
(4) adding to the CIP process a cleaning composition, an antimicrobial composition, or both.

2. The method of claim 1, further comprising:
rinsing the equipment with water to remove the cleaning composition or the antimicrobial composition.

3. The method of claim 1, wherein the antimicrobial composition comprises one or more antimicrobial agents selected from the group consisting of peroxycarboxylic acids, phenolic, halogen compound, quaternary ammonium compound, metal derivative, amine, alkanol amine, nitro derivative, analide, organosulfur, sulfur-nitrogen compounds, and combinations thereof.

4. The method of claim 1, wherein the enzyme composition comprises one or more enzymes selected from the group consisting of peroxidase, oxidoreductase, transferase, hydrolase, lyase, isomerase, ligase, protease, peptidase, lipase, esterase, amylase, polysaccharidase, carbohydrase, cellulase, hemicellulose, chitinase, glucanase, glycosidase, glucosidase, xylanase, mannanase, arabanase, DNase, RNase, phosphatase, phosphodiesterase, laccase, oxidoreductase, and combinations thereof.

5. The method of claim 1, wherein the enzyme composition further comprises one or more ingredients selected from the group consisting of enzyme stabilizer, salt, buffering agent, solvent, thickener, humectant, enzyme inhibitor, preservative, surfactant, dispersant, chelating agent, enzyme activity enhancer, and combinations thereof.

6. The method of claim 1, wherein the enzyme composition has a total enzyme amount from about 0.01% to about 10% by weight of the enzyme composition.

7. The method of claim 1, wherein the added enzyme composition in the CIP process forms a use solution that has a total enzyme amount in a range from about 10 ppm to about 1000 ppm.

8. The method of claim 1, wherein the CIP process is operated at a temperature of about 30° C. to about 85° C. in the presence of the enzyme composition.

9. The method of claim 1, wherein the enzyme composition added to the CIP process forms a use solution that has a pH value from about 2 to about 12.

10. The method of claim 1, wherein the cleaning composition is an acid cleaning composition.

11. The method of claim 1, wherein the cleaning composition is an alkaline cleaning composition.

12. The method of claim 1, wherein the cleaning composition includes an alkalinity source in an amount from about 0.01% to about 99% by weight of the cleaning composition.

13. The method of claim 1, wherein the fluid of the CIP process has a total amount of alkalinity source from about 5 ppm to about 25,000 ppm in the presence of the cleaning composition.

14. The method of claim 1, wherein the cleaning composition further comprises one or more antimicrobial agents selected from the group consisting of peroxycarboxylic acids, phenolic, halogen compound, quaternary ammonium compound, metal derivative, amine, alkanol amine, nitro derivative, anilide, organosulfur, sulfur-nitrogen compounds, and combinations thereof.

15. The method of claim 1, wherein the cleaning composition comprises one or more additives selected from the group consisting of oxidizing agent, chelating agent, sequestering agent, antimicrobial agent, dye, rheological modifier, gelling agent, thickener, pH modifiers, acid, base, preservative, processing aid, corrosion inhibitor, surfactant, dispersant, wetting agent, and combinations thereof.

16. The method of claim 1, wherein the method results in at least 1 log reduction of the microorganism population of the equipment compared to the microorganism population before adding the enzyme composition.

17. The method of claim 1, further comprising:
analyzing a sample of the fluid within the CIP process before, after, or before and after the addition of the enzyme composition to detect microorganisms within the fluid.

18. The method of claim 17, wherein the analyzing a sample of the enzyme solution comprises using an in-plate culture identification method.

19. The method of claim 1, wherein the culture identification method comprises cultivating the sample on a culture medium in a plate and performing a plate count for one or more microorganism, wherein the microorganism is selected from the group consisting of bacteria, yeast and mold, psychrophiles, psychrotrophs, mesopiles, thermophiles, spore, spoilage microorganism, pathogens, specific biofilm indicator microorganism, and combinations thereof.

20. The method of claim 17, wherein the analyzing a sample of the enzyme solution comprises using an ATP photometry method.

21. The method of claim 20, wherein the ATP photometry method comprises measuring the ATP level of a sample using bioluminescence assay.

22. The method of claim 20, wherein the ATP photometry method comprises:
extracting adenosine monophosphate (AMP) or adenosine diphosphate (ADP) from the sample; and
converting the AMP or the ADP to ATP using one or more enzymes; and
measuring the level of the converted ATP.

23. The method of claim 17, wherein the analyzing a sample of the enzyme solution comprises using one or more rapid microbial detection methods selected from the group consisting of: a PCR-based rapid detection technique; a biosensor; an immunoassay; a bioburden test; cell staining and subsequent optical quantification technique; extracellular polymeric substances (EPS) staining and subsequent optical quantification technique, and combinations thereof.

24. The method of claim 17, wherein the analyzing a sample of the enzyme solution comprises one or more steps selected from the group consisting of measuring oxygen consumption, measuring CO₂ production, measuring production of metabolites, and combinations thereof.

25. The method of claim 17, further comprising a remediation step in response to any detected microorganism, wherein the remediation step is selected from the group consisting of adjusting the concentration of the CIP composition, changing the chemistry of the CIP composition, adding additional CIP compositions, adding a cleaning composition to the CIP process, adding an antimicrobial composition to the CIP process, and combinations thereof.

26.-112. (canceled)
